SEQUENCE LISTING (14 sequences)

SEQ ID NO: 1 - protein, 34 aa, Artificial Sequence
  Chemically Synthesized
XLGPQGPPHLVADPSKKQGPWLEEEEEAYGWMDF

SEQ ID NO: 2 - protein, 34 aa, Artificial Sequence
  Chemically Synthesized
XLGPQGPPHLVADPSKKQGPWLEEEEEAYGWLDF

SEQ ID NO: 3 - protein, 17 aa, Artificial Sequence
  Chemically Synthesized
XGPWLEEEEEAYGWMDF

SEQ ID NO: 4 - protein, 17 aa, Artificial Sequence
  Chemically Synthesized
XGPWLEEEEEAYGWLDF

SEQ ID NO: 5 - protein, 101 aa, Artificial Sequence
  Chemically Synthesized
MQRLCVYVLIFALALAAFSEASWKPRSQQPDAPLGTGANRDLELPWLEQQGPASHHRRQLGPQGPPHLVADPSKKQGPWLEEEEEAYGWMDFGRRSAEDEN

SEQ ID NO: 6 - protein, 52 aa, Artificial Sequence
  Chemically Synthesized
DLELPWLEQQGPASHHRRQLGPQGPPHLVADPSKKQGPWLEEEEEAYGWMDF

SEQ ID NO: 7 - protein, 14 aa, Artificial Sequence
  Chemically Synthesized
WLEEEEEAYGWMDF

SEQ ID NO: 8 - protein, 6 aa, Artificial Sequence
  Chemically Synthesized
YGWMDF

SEQ ID NO: 9 - protein, 6 aa, Artificial Sequence
  Chemically Synthesized
YGWLDF

SEQ ID NO: 10 - protein, 6 aa, Artificial Sequence
  Chemically Synthesized
YGWMDF

SEQ ID NO: 11 - protein, 6 aa, Artificial Sequence
  Chemically Synthesized
YGWLDF

SEQ ID NO: 12 - protein, 10 aa, Artificial Sequence
  Chemically Synthesized
GAGAGAGAGA

SEQ ID NO: 13 - protein, 4 aa, Artificial Sequence
  Chemically Synthesized
WMNF

SEQ ID NO: 14 - protein, 4 aa, Artificial Sequence
  Chemically Synthesized
WLNF

(19) United States
(12) Patent Application Publication
(10) Pub. No.: US 2009/0054314 A1
(43) Pub. Date: Feb. 26, 2009

(54) Combined use of DPP-IV inhibitors and gastrin compounds

(76) Inventor: Antonio Cruz, Toronto (CA)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/545,025

(22) Filed: Oct. 6, 2006

Related U.S. Application Data
(60) Provisional application No. 60/724,919, filed on Oct. 7, 2005.

(51) Int. Cl. A61K 38/22; A61K 38/10; A61P 3/10

(52) U.S. Cl. 514/12; 514/13

(57) ABSTRACT

The invention relates to compositions, conjugates, and methods for the prevention and/or treatment of a condition and/or disease comprising a therapeutically effective amount of a DPP-IV inhibitor and a gastrin compound. The combination of a DPP-IV inhibitor and a gastrin compound provides beneficial effects, in particular sustained beneficial effects, in the prevention and/or treatment of conditions and/or diseases for which either a DPP-IV inhibitor or a gastrin compound have been demonstrated to have a therapeutic effect, including but not limited to diabetes, hypertension, chronic heart failure, fluid retentive states, obesity, metabolic syndrome and related diseases and disorders. Combinations of a DPP-IV inhibitor and a gastrin compound can be selected to provide unexpectedly additive effects or synergistic effects.

### RELATED APPLICATION

This application claims the benefit of U.S. Ser. No. 60/724,919, filed Oct. 7, 2005, which is incorporated herein by reference in its entirety.

### FIELD OF THE INVENTION

The invention relates generally to compositions, conjugates, and methods comprising a dipeptidylpeptidase-IV (DPP-IV) inhibitor and a gastrin compound, and uses thereof. In particular, the invention relates to a combination, (e.g., a combined preparation or pharmaceutical composition) which comprises a DPP-IV inhibitor and a gastrin compound for simultaneous, separate or sequential use, especially in the prevention, delay of progression or treatment of conditions for which either a DPP-IV inhibitor or gastrin compound have a therapeutic effect; the use of such combination for the preparation of a pharmaceutical composition for the prevention, delay of progression or treatment of such conditions; and, a method of prevention, delay of progression or treatment of such conditions.

### BACKGROUND OF THE INVENTION

Non-insulin dependent diabetes mellitus or (type 2 diabetes) is characterized by increased peripheral insulin resistance and abnormal insulin secretion. Glucose, amino-acids and gastrointestinal peptides are known to stimulate insulin secretion. Inhibitors of the dipeptidylpeptidase-IV (DPP-IV) enzyme are under investigation as drugs that may be useful in the treatment of diabetes, and particularly Type 2 diabetes. DPP-IV acts in vivo by inactivating incretins such as glucagon like peptide-1 (GLP-1) and gastric inhibitory peptide (GIP). GLP-1 and GIP are produced when food is consumed and they stimulate production of insulin. Inhibition of DPP-IV leads to decreased inactivation of the incretins, resulting in increased effectiveness of the incretins to stimulate insulin. Inhibition of DPP-IV therefore results in increased levels of serum insulin in a subject.

### SUMMARY OF THE INVENTION

Disclosed herein is the combination of a DPP-IV inhibitor and a gastrin compound for use in the prevention and/or treatment of conditions and/or diseases for which either a DPP-IV inhibitor or a gastrin compound have a therapeutic effect, including but not limited to diabetes, more particularly Type II diabetes mellitus, conditions of impaired glucose tolerance (IGT), conditions of impaired fasting plasma glucose, metabolic acidosis, ketosis, arthritis, hypertension, chronic heart failure, fluid retentive states, obesity, metabolic syndrome obesity, dyslipidemia, osteoporosis, and related diseases and disorders. Combinations of a DPP-IV inhibitor and a gastrin compound may be selected to provide additive effects or greater than additive effects, i.e. synergistic effects.

The therapeutic strategy of the present invention relates to the modification of multiple pathophysiological processes using innovative combinations of compounds that have distinct complementary, additive or synergistic mechanisms of action to provide safe and effective treatments for conditions and/or diseases disclosed herein. Compositions, conjugates, or methods comprising at least one DPP-IV inhibitor and at least one gastrin compound employing different mechanisms to achieve maximum therapeutic efficacy, may improve tolerance to the therapy with a reduced risk of side effects that may result from higher doses or longer term monotherapies (i.e. therapies with each compound alone). A composition, conjugate, or method of the invention may permit the use of lower doses of one or both compounds with reduced adverse toxic effects of each compound. A suboptimal dosage may provide an increased margin of safety, and may also reduce the cost of a drug necessary to achieve prophylaxis and therapy. In certain aspects of the invention, the increased convenience of a single combination dosage unit may result in enhanced compliance. Other advantages of a composition, conjugate, or combination therapy may include higher stability towards degradation and metabolism, longer duration of action, and/or longer duration of action or effectiveness at particularly low doses.

Broadly stated, the invention relates to compositions, conjugates, and methods for the prevention and/or treatment of a condition and/or disease comprising at least one DPP-IV inhibitor and at least one gastrin compound.

In an aspect, the invention relates to compositions, conjugates, and methods for the prevention and/or treatment of a condition and/or disease disclosed herein comprising a therapeutically effective amount of at least one DPP-IV inhibitor and at least one gastrin compound that provide one or more beneficial effects.

In an aspect, the invention relates to compositions, conjugates, and methods for the prevention and/or treatment of diabetes in a subject receiving insulin comprising therapeutically effective amounts of at least one DPP-IV inhibitor and at least one gastrin compound that provide one or more beneficial effects.

A composition, conjugate, or method of the invention may provide beneficial effects in particular sustained beneficial effects following treatment or termination of treatment. Beneficial effects may be evidenced by increased C-peptide production, increases in pancreatic insulin production, and/or about normal blood glucose levels compared with a DPP-IV inhibitor or gastrin compound alone.

In an aspect, the invention contemplates a composition, preferably a pharmaceutical composition, comprising therapeutically effective amounts of at least one DPP-IV inhibitor and at least one gastrin compound. In another aspect the invention provides a pharmaceutical composition comprising at least one DPP-IV inhibitor and at least one gastrin compound that provide beneficial effects, preferably sustained beneficial effects, following treatment. A pharmaceutical composition may optionally comprise a pharmaceutically acceptable carrier, excipient, or vehicle.

In another aspect, the invention relates to a combination, such as a combined preparation or pharmaceutical composition, comprising at least one DPP-IV inhibitor and at least one gastrin compound, and at least one additional pharmaceutically acceptable carrier, excipient, or vehicle.

The invention also contemplates a pharmaceutical composition in separate containers and intended for simultaneous or sequential administration preferably to provide beneficial effects, more preferably sustained beneficial effects, comprising a DPP-IV inhibitor and a gastrin compound, both optionally together with pharmaceutically acceptable carriers, excipients, or vehicles.

The invention further contemplates a conjugate comprising a DPP-IV inhibitor interacting with or linked to a gastrin compound preferably to provide beneficial effects, more preferably sustained beneficial effects.

In an aspect the invention relates to a medicinal combination of active principles, having, jointly, a complementary and/or synergistic action, this being for the treatment of diabetes, in particular of Type I diabetes.

In an aspect the invention relates to a medicinal combination of active principles, having, jointly, a complementary and/or synergistic action, this being for the treatment of diabetes, in particular of Type II diabetes.

The invention still further contemplates methods for preparing compositions and conjugates of the invention that result in compositions and conjugates preferably with beneficial effects, more preferably sustained beneficial effects.

In an aspect of the invention, a method is provided for preparing a stable pharmaceutical composition of a DPP-IV inhibitor and a gastrin compound preferably adapted to provide beneficial effects, more preferably sustained beneficial effects, following treatment, comprising preparing a composition comprising the DPP-IV inhibitor, a gastrin compound, and a pharmaceutically acceptable carrier, excipient, or vehicle preferably effective to physically stabilize the DPP-IV inhibitor.

In another aspect of the invention, a method is provided for preparing a stable pharmaceutical composition of a DPP-IV inhibitor comprising mixing a DPP-IV inhibitor, a gastrin compound, and a pharmaceutically acceptable carrier, excipient, or vehicle effective to physically stabilize the DPP-IV inhibitor and preferably adapted to provide beneficial effects, more preferably sustained beneficial effects.

The invention relates to a combination treatment for preventing and/or treating a condition and/or disease discussed herein in a subject comprising administering to the subject a therapeutically effective amount of at least one DPP-IV inhibitor and a gastrin compound to preferably provide beneficial effects. In an aspect the invention provides a combination treatment or intervention which provides sustained beneficial effects following treatment.

The invention further relates to the use of a DPP-IV inhibitor and a gastrin compound, a composition, or conjugate of the invention for preventing, and/or ameliorating disease severity, disease symptoms, and/or periodicity of recurrence of a condition and/or disease described herein. The invention still further relates to the prevention and/or treatment, in a subject, of diseases and/or conditions using a DPP-IV inhibitor and a gastrin compound, a composition, or conjugate of the invention.

In an aspect, the invention provides a method for the prevention and/or intervention of a condition and/or disease discussed herein in a subject comprising administration of at least one DPP-IV inhibitor and at least one gastrin compound, or a composition or conjugate of the invention. A DPP-IV inhibitor and a gastrin compound, composition or conjugate may be directly administered to a subject or contacted with cells (e.g. stem cells or progenitor cells) and administered to a subject.

The invention provides in some aspects methods for the potentiation of a gastrin compound in the treatment of a condition and/or disease in a subject, in particular diabetes and related diseases, disorders, or conditions, comprising co-administering at least one gastrin compound and at least one DPP-IV inhibitor to the subject.

The invention provides in some aspects methods for the potentiation of a DPP-IV inhibitor in the treatment of a condition and/or disease in a subject, in particular diabetes and related diseases, disorders, or conditions, comprising co-administering one or both of at least one gastrin compound and at least one DPP-IV inhibitor to the subject.

In other aspects, the invention provides a method for the prevention and/or intervention of a condition and/or disease discussed herein in a subject comprising administration of at least one DPP-IV inhibitor and at least one gastrin compound to a subject in need thereof to provide beneficial effects.

In another aspect, the invention provides a method for the prevention and/or intervention of a condition and/or disease discussed herein in a subject comprising co-administering at least one DPP-IV inhibitor and at least one gastrin compound to a subject in need thereof.

In a particular aspect, the invention relates to inducing islet neogenesis in a subject comprising contacting islet precursor cells with a DPP-IV inhibitor and a gastrin compound, composition, or conjugate of the invention in a sufficient amount to increase proliferation of islet precursor cells in the subject thereby inducing islet neogenesis.

In another aspect, the invention relates to a method for treating diabetes mellitus, in particular Type II diabetes, in a patient in need thereof by administering a gastrin compound and a DPP-IV inhibitor or a composition comprising a gastrin compound and a DPP-IV inhibitor in an amount sufficient to effect differentiation of the patient's pancreatic islet precursor cells to mature insulin-secreting cells and/or to stimulate insulin synthesis in existing islet cells.

In an embodiment, the invention relates to methods of increasing, preserving, or reducing rate of loss in insulin secretion in a subject comprising administration of therapeutically effective amounts of a DPP-IV inhibitor and a gastrin compound, composition, or conjugate of the invention to a subject in need thereof.

In another embodiment, the invention relates to methods of increasing, preserving, or reducing rate of loss of β-cell function in a subject comprising administration of therapeutically effective amounts of a DPP-IV inhibitor and a gastrin compound, composition, or conjugate of the invention to a subject in need thereof.

In another embodiment, the invention relates to methods of increasing, preserving, or reducing rate of loss in number and/or size of β-cells in a subject comprising administration of therapeutically effective amounts of a DPP-IV inhibitor and a gastrin compound, composition, or conjugate of the invention to a subject in need thereof.

In an embodiment, the invention relates to treatment of diseases benefiting from an increase, preservation, or reduction in rate of loss in insulin secretion in a subject comprising administration of therapeutically effective amounts of a DPP-IV inhibitor and a gastrin compound, composition, or conjugate of the invention to a subject in need thereof.

In an embodiment, the invention relates to treatment of diseases benefiting from an increase, preservation, or reduction in rate of loss of β-cell function in a subject comprising administration of therapeutically effective amounts of a DPP-IV inhibitor and a gastrin compound, composition, or conjugate of the invention to a subject in need thereof.

In an embodiment, the invention relates to treatment of diseases benefiting from an increase, preservation, or reduction in rate of loss in number and/or size of β-cells in a subject comprising administration of therapeutically effective amounts of a DPP-IV inhibitor and a gastrin compound, composition, or conjugate of the invention to a subject in need thereof.

The invention provides methods for treating cells using a DPP-IV inhibitor and a gastrin compound, or compositions, or conjugates of the invention. In particular, the invention relates to a method for expanding and differentiating stem cells or progenitor cells into insulin secreting cells, enhancing proliferation of insulin secreting cells, and/or sustaining islet cells or precursor cells. Cells may be contacted with a DPP-IV inhibitor and a gastrin compound in culture or in a subject.

In an aspect, a method is provided for treating a condition and/or disease comprising administering a DPP-IV inhibitor and a gastrin compound, a composition or conjugate of the invention, with a plurality of cells to a subject in need thereof preferably to thereby produce beneficial effects, more preferably sustained beneficial effects. In an embodiment, the compounds/composition/conjugate are administered systemically.

In another aspect, the invention provides a method for treating a subject with a condition and/or disease discussed herein comprising contacting ex vivo a plurality of cells with a DPP-IV inhibitor and a gastrin compound, or a composition or conjugate of the invention, optionally culturing the cells, and administering the cells to the subject in need thereof.

Also provided in particular aspects of the invention are methods and compositions for treating diabetes in a patient in need thereof by implanting into a diabetic patient pancreatic islet cells that have been exposed in culture to a sufficient amount of a gastrin compound and a DPP-IV, or a composition or conjugate of the invention, to increase the number of pancreatic beta cells in the islets; optionally the population of pancreatic beta cells can be grown in culture for a time sufficient to expand the population of β-cells prior to transplantation.

The invention also contemplates the use of a composition comprising a combination of at least one DPP-IV inhibitor and at least one gastrin compound for the preparation of one or more medicament for preventing and/or treating a condition and/or disease. The invention further contemplates use of a DPP-IV inhibitor in combination with a gastrin compound for the manufacture of a medicament for the treatment of a condition and/or disease. Still further the invention provides use of a DPP-IV inhibitor for the manufacture of a medicament for the treatment of a condition and/or disease to be used in combination with a gastrin compound. In addition the invention provides use of a DPP-IV inhibitor in combination with a gastrin compound for the manufacture of a medicament for increasing, preserving or reducing loss of insulin secretion, loss of β-cell function, or number and or size of β-cells in a subject.

In an aspect, the invention relates to the use of additive, complementary, or synergistically effective amounts of at least one DPP-IV and at least one gastrin compound for the preparation of a medicament for preventing or treating a condition and/or disease. In another aspect, the invention relates to the use of a DPP-IV inhibitor and a gastrin compound for the preparation of a medicament which has a protracted profile of action. The invention additionally provides uses of a pharmaceutical composition and a conjugate of the invention in the preparation of medicaments for the prevention and/or treatment of conditions and/or diseases. The medicaments provide beneficial effects, preferably sustained beneficial effects following treatment.

The invention also relates to the use of a combination or composition of the invention for the cosmetic treatment of a subject in order to affect a cosmetically beneficial loss of body weight, and a method of improving the bodily appearance of a subject.

Compositions or methods described herein may optionally include other agents such as antidiabetic compounds, immunosuppressive agents, antiobesity agents, antidiabetic agents, appetite regulating drugs, antihypertensive agents, and agents for the treatment and/or prevention of complications resulting from or associated with a condition and/or disease. In an aspect of the invention, the other agent is a PPAR compound, and a therapeutically effective amount of a PPAR compound is administered separately or together with a DPP-IV inhibitor and a gastrin compound.

Since the present invention relates to a method of prevention and/or treatment comprising a combination of active agents which may be administered separately or as conjugates, the invention also provides a kit comprising a DPP-IV inhibitor and a gastrin compound, and a pharmaceutical composition, or conjugate of the invention in kit form. In an aspect, the invention provides a method of promoting sales of a composition or kit of the invention comprising the public distribution of information that administration of the composition or kit is associated with β-cell proliferation and/or islet neogenesis.

These and other aspects, features, and advantages of the present invention should be apparent to those skilled in the art from the following detailed description.

### DETAILED DESCRIPTION OF THE INVENTION

### Glossary

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, it is to be understood that “a,” “an,” and “the” include plural referents unless the content clearly dictates otherwise. Thus, for example, reference to “a compound” includes a mixture of two or more compounds. Thus, the phrase “a DPP-IV inhibitor”, as used herein can also mean “one or more DPP-IV inhibitor” or “at least one DPP-IV inhibitor”. The phrase “a gastrin compound”, as used herein can also mean “one or more gastrin compound” or “at least one gastrin compound”.

The term “about” means plus or minus 0.1 to 50%, 5-50%, or 10-40%, preferably 10-20%, more preferably 10% or 15%, of the number to which reference is being made.

Selected compounds described herein contain one or more asymmetric centers and may give rise to enantiomers, diasteriomers, and other stereoisomeric forms which may be defined in terms of absolute stereochemistry as (R)- or (S)-. Therefore, the invention includes all such possible diasteriomers and enantiomers as well as their racemic and optically pure forms. Optically active (R)- and (S)-isomers may be prepared using chiral synthons or chiral reagents, or resolved using conventional techniques. When the compounds described herein contain centers of geometric asymmetry, and unless specified otherwise, it is intended that the compounds include both E and A geometric isomers. All tautomeric forms are intended to be included within the scope of the invention.

The terms “subject”, “individual” or “patient” refer to an animal including a warm-blooded animal such as a mammal, which is afflicted with or suspected of having or being pre-disposed to a condition and/or disease disclosed herein. Preferably, the terms refer to a human. The terms also include domestic animals bred for food, sport, or as pets, including horses, cows, sheep, poultry, fish, pigs, cats, dogs, and zoo animals. The methods herein for use on subjects/individuals/patients contemplate prophylactic as well as curative use. Typical subjects for treatment include persons susceptible to, suffering from or that have suffered a condition and/or disease discussed herein.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. The use of such media and agents for an active substance is well known in the art. In certain aspects of the invention, a carrier, excipient, or vehicle is selected to stabilize a DPP-IV inhibitor and/or a gastrin compound.

“Pharmaceutically acceptable salt(s),” means a salt that is pharmaceutically acceptable and has the desired pharmacological properties. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Suitable salts include salts that may be formed where acidic protons in the compounds are capable of reacting with inorganic or organic bases. Suitable inorganic salts include those formed with alkali metals, e.g. sodium and potassium, magnesium, calcium, and aluminium. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benezenesulfonic acid). When there are two acidic groups present, a pharmaceutically acceptable salt may be a mono-acid-mono-salt or a di-salt; and similarly where there are more than two acidic groups present, some or all of such groups can be salified.

The terms “preventing and/or treating”, “prevention and/or treatment”, or “prevention and/or intervention” refer to the administration to a subject of biologically active agents either before or after onset of a condition and/or disease. A treatment may be either performed in an acute or chronic way. In particular, prevention includes the management and care of a subject at risk of developing a condition and/or disease discussed herein prior to the clinical onset of the condition and/or disease. Treatment or intervention refers to the management and care of a subject at diagnosis or later. An objective of prevention, treatment, or intervention is to combat the condition and/or disease and includes administration of the active compounds to prevent or delay the onset of the symptoms or complications, or alleviating the symptoms or complications, or eliminating or partially eliminating the condition and/or disease.

A “beneficial effect” refers to an effect of a combination of a DPP-IV inhibitor and a gastrin compound, or composition or conjugate thereof, that differs or is greater than the effect of either of the compounds alone. The beneficial effect includes favorable pharmacological and/or therapeutic effects, and improved pharmacokinetic properties and/or biological activity. A beneficial effect may be a complementary effect, an additive effect or synergistic effect. In preferred embodiments of the invention, beneficial effects include but are not limited to the following: reduced or absent islet inflammation, decreased disease progression, increased survival, or elimination or partial elimination of a condition and/or disease. In a more preferred embodiment, the beneficial effect is a “sustained beneficial effect” where the beneficial effect is sustained for a prolonged period of time after termination of treatment. In an embodiment, one or more of the aforementioned effects are sustained for a prolonged period of time after termination of treatment. A beneficial effect may be sustained for at least about 2, 4, 6, 8, 10, 2 to 4 weeks, 2 to 6 weeks, 2 to 8 weeks, 2 to 12 weeks, 2 to 24 weeks, 2 weeks to 12 months, and 2 weeks to 18 months following treatment. The period of time a beneficial effect is sustained may correlate with the duration and timing of the treatment. A subject may be treated continuously for about 2 to 8 weeks, 2 to 12 weeks, 2 to 16 weeks, 2 weeks to 6 months, 2 weeks to 12 months, or periodically. A sustained beneficial effect may manifest as one or more of increased C-peptide production, increased pancreatic insulin production, about normal or low blood glucose levels for a prolonged period following treatment, increased beta cell production and/or inhibition of programmed cell death (apoptosis), or reduction in insulin use in a subject.

The beneficial effect may be a statistically significant effect in terms of statistical analysis of an effect of the two compounds versus the effects of each of the compounds. “Statistically significant” or “significantly different” effects or levels with two compounds compared with each compound alone may represent levels that are higher or lower than a standard. In embodiments of the invention, the difference may be 1.5, 2, 3, 4, 5, or 6 times higher or lower compared with the effect obtained with each compound alone.

An “additive effect” of a DPP-IV inhibitor and a gastrin compound refers to an effect that is equal to the sum of the effects of the two individual compounds

A “synergistic effect” of a DPP-IV inhibitor and a gastrin compound refers to an effect that is greater than the additive effect which results from the sum of the effects of the two individual compounds.

“Combination treatment”, “combination therapy”, and “administering in combination” are used interchangeably herein and mean that the active ingredients are administered concurrently to a patient being treated. When administered in combination each component may be administered at the same time, or sequentially in any order at different points in time. Therefore, each component may be administered separately, but sufficiently close in time to provide the desired effect, in particular a beneficial, more particularly an additive, or synergistic effect. The first compound may be administered in a regimen which additionally comprises treatment with the second compound. In certain embodiments, the term refers to administration of a DPP-IV inhibitor and a gastrin compound to a patient within one year, including separate administration of two medicaments each containing one of the compounds as well as simultaneous administration whether or not the two compounds are combined in one formulation or whether they are two separate formulations.

A “medicament” refers to a pharmaceutical composition suitable for administration of a pharmaceutically active compound(s) (e.g. a DPP-IV inhibitor and/or a gastrin compound) to a patient.

“Therapeutically effective amount” relates to the amount or dose of active compounds (e.g. DPP-IV inhibitor or gastrin compound), compositions or conjugates of the invention that will lead to one or more desired beneficial effects, preferably one or more sustained beneficial effects. A “therapeutically effective amount” can provide a dosage which is sufficient in order for prevention and/or treatment of a subject to be effective compared with no treatment.

“Synergistically effective amount” relates to the amount, dosage, or dose of active compounds (e.g. DPP-IV inhibitor and gastrin compound), compositions or conjugates of the invention that will provide a synergistic effect, in particular a synergistic beneficial effect.

The expression “complementary action” or “complementary effect” refers to the pharmacological action of one, two or more different compounds making it possible to act on the same pathology via different pharmacological mechanisms, for example the combined use of at least one DPP-IV inhibitor and at least one gastrin compound.

The term “potentiation” refers to an increase of a corresponding pharmacological activity or therapeutic effect. Potentiation of one component of a combination or composition of the present invention by co-administration of the other components according to the present invention means that an effect is being achieved that is greater than that achieved with one component alone.

“Suboptimal dose” or “suboptimal dosage” refers to an amount, dose or dosage of an active compound which is less than the optimal amount, dose or dosage for that compound when used in monotherapy.

The terms “associated”, “linked”, “interact”, “interaction”, or “interacting” refer to any physical association between molecules. The terms preferably refer to a stable association between two molecules due to, for example, electrostatic, hydrophobic, ionic, hydrogen-bond interactions, or covalent interactions.

In the present context, a “DPP-IV inhibitor” is an antagonist of a dipeptidylpeptidase-IV (DPP-IV) or another member of the family of serine peptidases that includes quiescent cell proline dipeptidase, DPP8, and DPP9. A DPP-IV inhibitor may exhibit inhibition of the enzymatic activity of DPP IV and functionally related enzymes, such as from 1-100% inhibition, and especially preserve the action of substrate molecules, including but not limited to GLP-1, GIP, peptide histidine methionine, and other similar molecules. A DPP IV inhibitor may indirectly affect the levels of GLP-1 (Hughes, T. et al., 2002, Am I Diabetes Assoc Abstract 272) by inhibiting an enzyme involved in its integrity. A DPP-IV inhibitor can be a peptide or a non-peptide compound; in particular the DPP-IV inhibitor is a non-peptide compound.

The term “DPP-IV inhibitor” is also intended to comprise active metabolites and prodrugs of a DPP-IV inhibitor, such as active metabolites and prodrugs of DPP-IV inhibitors. A “metabolite” refers to an active derivative of a DPP-IV inhibitor produced when the DPP-IV inhibitor is metabolized. A “prodrug” refers to a compound that is either metabolized to a DPP-IV inhibitor or is metabolized to the same metabolite(s) as a DPP-IV inhibitor. The inhibitors also include corresponding stereoisomers as well as polymorphs, e.g., crystal modifications.

Representative DPP-IV inhibitors are listed in Table 1. DPP-IV inhibitors are in each case generically and specifically disclosed in the referenced patent document or publication. Any of the substances disclosed in the patent documents and publications referenced in Table 1 (and the documents referenced therein) is considered potentially useful as DPP-IV inhibitors to be used in carrying out the present invention.

Specific examples of DPP-IV inhibitors include, but are not limited to, dipeptide derivatives or dipeptide mimetics such as alanine-pyrrolidide, isoleucine-thiazolidide, and the pseudosubstrate N-valyl prolyl, O-benzoyl hydroxylamine.

In particular aspects of the invention, the DPP-IV inhibitor is Sitagliptin (Januvia®) [Merck & Co.], Vildagliptin (LAF 237, Galvus®) [Novartis], PSN9301, Saxagliptin (BMS-477118) [Bristol Myers Squibb], N-(N′-substituted glycyl)-2-cyanopyrrolidines, L-threo-isoleucyl thiazolidine, L-allo-isoleucyl thiazolidine, L-threo-isoleucyl pyrrolidine, isoleucine thiazolidide, valine purrolidide, NVP-DPP738 (Novartis, Cambridge, Mass.), P32/98 (Probiodrug AG, Halle, Germany), P93/01 (Probiodrug), and L-allo-isoleucyl pyrrolidine described in U.S. Pat. No. 6,001,155, WO 99/61431, WO 99/67278, WO 99/67279, DE 198 34 591, WO 97/40832, DE 196 16 486 C₂, WO 98/19998, WO 00/07617, WO 99/38501, and WO 99/46272, and US20050222221, and optionally in any case pharmaceutical salts thereof.

Particular DPP-IV inhibitors for the combinations, uses, methods, and kits of the present invention are 1-{2-[(5-cyanopyridin-2-yl) amino]ethylamino}acetyl-2 (S)— cyano-pyrrolidine dihydrochloride (DPP728), especially the dihydrochloride thereof; (S)-1-[(3-hydroxy-1-adamantyl)amino]acetyl-2-cyano-pyrrolidine (Vildagliptin Galvus®, LAF237); L-threo-isoleucyl thiazolidine (compound code according to Probiodrug: P32/98); MK-0431; GSK23A; Sitagliptin (Januvia®) [Merck & Co.], Saxagliptin (BMS-477118) [Bristol Myers Squibb]; 3-(aminomethyl)-2-isobuthyl-1-oxo-4-phenyl-1,2-dihydro-6-isoquinoline carboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

In an aspect of the invention, the DPP-IV inhibitor is Sitagliptin (Januvia®) [Merck & Co.].

In one aspect of the invention, the DPP-IV inhibitor is valine-pyrrolidide.

In another aspect of the present invention, the DPP-IV inhibitor is 3-(L-Isoleucyl)thiazolidine (isoleucine-thiazolidide).

In another aspect of the present invention, the DPP-IV inhibitor is 1-[2-[5-cyanopyridin-2-yl)amino]ethylamino]acetyl-2-cyano-(S)-pyrrolidine (NVP-DPP728).

In another aspect of the present invention, the DPP-IV inhibitor is 3(R)-Amino-1-[3-(trifluoromethyl)-5,6,7,8-tetrahydro[1,2,4]triazolo[4,3-a]pyrazin-7-yl]-4-(2,4,5-trifluorophenyl)butan-1-one (MK-0431).

In another aspect of the present invention, the DPP-IV inhibitor is (1S,3S,5S)-2-[2(S)-Amino-2-(3-hydroxyadamantan-1-yl)acetyl]-2-azabicyclo[3.1.0]hexane-3-carbonitrile (Saxagliptin, BMS-477118).

In another aspect of the present invention, the DPP-IV inhibitor is [1-[2(S)-Amino-3-methylbutyryl]pyrrolidin-2(R)-yl]boronic acid (PT-100).

In another aspect of the present invention, the DPP-IV inhibitor is GSK-823093; PSN-9301; T-6666; SYR-322; SYR-619; CR-14023; CR-14025; CR-14240; CR-13651; NNC-72-2138; NN-7201; PHX-1149; PHX-1004; SNT-189379; GRC-8087; PT-630; SK-0403; GSK-825964; TS-021; GRC-8200; GRC-8116; or FE107542.

In one aspect of the present invention, the DPP-IV inhibitor is (1-[β-hydroxy-1-adamantyl)amino]acetyl]-2-cyano-(S)-pyrrolidine (Vildagliptin, Galvus®, LAF237).

In an aspect of the present invention, the DPP-IV inhibitor is not a dipeptide derivative.

In an aspect of the present invention, the DPP-IV inhibitor is not a dipeptide mimetic.

In an aspect of the present invention, the DPP-IV inhibitor is not identical to valine-pyrrolidide.

In an aspect of the present invention, the DPP-IV inhibitor is not identical to alanine-pyrrolidide.

In an aspect of the present invention, the DPP-IV inhibitor is not identical to 3-(L-Isoleucyl)thiazolidine (isoleucine-thiazolidide).

In an aspect of the present invention, the DPP-IV inhibitor is not identical to N-valyl propyl, O-benzoyl hydroxylamine.

In an aspect of the present invention, the DPP-IV inhibitor is not identical to 1-[[[2-[(5-cyanopyridin-2-yl)amino]ethyl]amino]acetyl]-2-cyano-(S)-pyrrolidine (NVP-DPP728).

In an aspect of the present invention, the DPP-IV inhibitor is not identical to 3(R)-Amino-1-[3-(trifluoromethyl)-5,6,7,8-tetrahydro[1,2,4]triazolo[4,3-a]pyrazin-7-yl]-4-(2,4,5-trifluorophenyl)butan-1-an (MK-0431).

In an aspect of the present invention, the DPP-IV inhibitor is not identical to (1-[[3-hydroxy-1-adamantyl)amino]acetyl]-2-cyano-(S)-pyrrolidine (Vildagliptin, Galvus®, LAF237).

In an aspect of the present invention, the DPP-IV inhibitor is not identical to (1S,3S,5S)-2-[2(S)-Amino-2-(3-hydroxyadamantan-1-yl)acetyl]-2-azabicyclo[3.1.0]hexane-3-carbonitrile (BMS-477118).

In an aspect of the present invention, the DPP-IV inhibitor is not identical to [1-[2(S)-Amino-3-methylbutyryl]pyrrolidin-2(R)-yl]boronic acid (PT-100).

In an aspect of the present invention, the DPP-IV inhibitor is not identical to one or more of GSK-823093; PSN-9301; T-6666; SYR-322; SYR-619; CR-14023; CR-14025; CR-14240; CR-13651; NNC-72-2138; NN-7201; PHX-1149; PHX-1004; SNT-189379; GRC-8087; PT-630; SK-0403; GSK-825964; TS-021; GRC-8200; GRC-8116, and FE107542.

In an aspect of the present invention, any one or more DPP-IV inhibitor may be excluded from any embodiment of the present invention. A DPP-IV inhibitor can be selected that has an IC₅₀ of less than about 10 μM, less than about 1 μM, less than about 100 nM, less than about 75 nM, less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM. In an embodiment, a DPP-IV inhibitor is selected that has an IC₅₀ of less than about 50 nM, less than about 25 nM, less than about 20 nM, less than about 15 nM, less than about 10 nM, less than about 5 nM, less than about 4 nM, less than about 3 nM, less than about 2 nM, or less than about 1 nM. In an aspect of the present invention, a selective DPP-IV inhibitor is utilized that has a selectivity for human plasma DPP-IV over one or more of PPCE, DPP-II, DPP-8 and DPP-9 of at least about 10-fold, more preferably of at least about 100-fold, and most preferably of at least about 1000-fold. In an aspect of the present invention, the DPP-IV inhibitor is orally active. In particular aspects of the invention, the DPP-IV inhibitor is an inhibitor of human DPP-IV.

DPP-IV inhibitors can be prepared by a variety of methods known in the art.

The term “substantial similarity” or “substantial sequence similarity,” when referring to a nucleic acid or fragment thereof, or polypeptide indicates that, when optimally aligned with another nucleic acid or fragment or polypeptide there is a percent sequence identity in at least about 50%, more preferably 60% of the nucleotide bases or amino acid residues, usually at least about 70%, more usually at least about 80%, preferably at least about 90%, and more preferably at least about 95-98% of the nucleotide bases or amino acid residues, as measured by any well-known algorithm of sequence identity, such as FASTA, BLAST or Gap.

“Percent sequence identity” refers to the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA, Gap or Bestfit (Wisconsin Package Version 10.0, Genetics Computer Group (C4CG), Madison, Wis.; Pearson, Methods Enzymol. 183: 63-98, 1990; Pearson, Methods Mol. Bio. 276: 71-84, 1998). The BLAST programs are publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

An “analog” refers to a polypeptide wherein one or more amino acid residues of a parent or wild-type polypeptide have been substituted by another amino acid residue, one or more amino acid residues of a parent or wild-type polypeptide have been inverted, one or more amino acid residues of the parent or wild-type polypeptide have been deleted, and/or one or more amino acid residues have been added to the parent or wild-type polypeptide. Such an addition, substitution, deletion, and/or inversion may be at either of the N-terminal or C-terminal end or within the parent or wild-type polypeptide, or a combination thereof. Typically “an analog” is a peptide wherein 6 or less amino acids have been substituted and/or added and/or deleted from the parent or wild-type peptide, more preferably a peptide wherein 3 or less amino acids have been substituted and/or added and/or deleted from the parent or wild-type polypeptide, and most preferably, a peptide wherein one amino acid has been substituted and/or added and/or deleted from the parent or wild-type polypeptide.

Mutations may be introduced into a polypeptide by standard methods, such as site-directed mutagenesis and PCR-mediated mutagenesis. Conservative substitutions can be made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed.

A “derivative” refers to a polypeptide in which one or more of the amino acid residues of a parent polypeptide have been chemically modified. Derivatives may be obtained by chemically modifying one or more amino acid residues of the parent polypeptide or analog thereof, for instance by alkylation, acylation, glycosylation, pegylation, ester formation, deamidation, amide formation, or by introducing a lipophilic functionality.

A “chimeric polypeptide” comprises all or part (preferably biologically active) of a selected polypeptide operably linked to a heterologous polypeptide (i.e., a polypeptide other than the selected polypeptide). Within the fusion polypeptide, the term “operably linked” is intended to indicate that a selected polypeptide and the heterologous polypeptide are fused in-frame to each other. The heterologous polypeptide can be fused to the N-terminus or C-terminus of a selected polypeptide. Chimeric and fusion proteins can be produced by standard recombinant DNA techniques.

A “gastrin compound” refers to any compound, including peptides and non-peptide compounds, which fully or partially associate with and/or activate a gastrin/CCK receptor, in particular a gastrin/CCK_{B} receptor, and/or increase gastrin secretion.

A “gastrin/CCK receptor” refers to a member of the G-protein-coupled receptor family that displays a characteristic binding affinity for a cholecystokinin (CCK) including without limitation CCK-8, desulfated CCK-8, CCK-33, CCK-4, or gastrins including without limitation desulfated or sulfated gastrin-17, or pentagastrin, or other CCK or gastrin analogues or family members. Examples of CCK/gastrin receptor proteins are CCK_{A} and CCK_{B}/gastrin receptors, in particular a CCK_{B}/gastrin receptors.

In aspects of the invention, a gastrin compound is selected that has a suitable IC₅₀, for example an IC₅₀ of about ˜0.7 nM at a gastrin/CCK_{B} receptor, as measured by methods known in the art [see Singh et al (1995) J. Biol. Chem. 270: 8429-8438, and Kopin et al (1995) J. Biol. Chem. 270: 5019-5023 describing in vitro cell growth assays, and receptor binding assays as described in Singh et al (1995) J. Biol. Chem. 270: 8429-8438, and Kopin et al (1995) J. Biol. Chem. 270: 5019-5023]. A gastrin compound may also be selected based on other criteria such as activity, half-life etc. as discussed herein.

Gastrin compounds that may be used in the present invention include without limitation one or more of a gastrin compound including a gastrin agonist, a cholecystokinin, or a cholecytokinin agonist.

The term “gastrin compound” encompasses selected compounds that in combination with a DPP-IV inhibitor provide at least one beneficial effect. In other aspects of the invention a gastrin compound is selected to so that, in combination with a DPP-IV inhibitor, neogenesis of insulin-producing pancreatic islet cells is induced. In a further aspect the term includes any gastrin compound that demonstrates additive, synergistic, or complementary activity with a DPP-IV inhibitor.

The term includes analogs, derivatives, fragments and modifications of a wild-type gastrin and chimeric polypeptides comprising gastrin. In aspects of the invention a gastrin compound includes a polypeptide that shares substantial sequence similarity with a mammalian gastrin and possesses some or all of the biological activity of a mammalian gastrin. In certain aspects, a gastrin compound may be an active analog, fragment or other modification which, for example, share amino acid sequence similarity with an endogenous mammalian gastrin, for example, share 60%, 70%, 80%, 90%, 95%, 98%, or 99% identity.

Gastrin compounds may be synthesized by chemical synthesis using techniques well known in the chemistry of proteins such as solid phase synthesis (Merrifield, 1964, J. Am. Chem. Assoc. 85:2149-2154) or synthesis in homogenous solution (Houbenweyl, 1987, Methods of Organic Chemistry, ed. E. Wansch, Vol. 15 I and II, Thieme, Stuttgart). The synthesis may be performed using manual procedures or by automation. Automated synthesis may be carried out, for example, using an Applied Biosystems 431A peptide synthesizer (Perkin Elmer). Gastrin compounds may also be obtained from commercial sources. For example, synthetic human gastrin 17 with methionine or leucine at position 15 is available from Bachem AG, Bubendorf, (Switzerland), and from Research Plus Inc (New Jersey, USA).

A “gastrin compound” includes, without limitation, the various forms of gastrin, such as gastrin 71 (Accession Nos. AAH69762 and NP_{—}000796), gastrin 52, gastrin 34 (big gastrin), gastrin 17 (little gastrin), gastrin 14, and gastrin 8 (mini gastrin), pentagastrin, tetragastrin, and fragments, analogs, and derivatives thereof. Sequences for gastrins including big gastrin-34 (Bonato et al, 1986, Life Science 39:959) and small gastrin-17 (Bentley et al (1966) Nature 209:583) are known in the art, and some are shown in SEQ ID NOs. 1 to 9. In particular, sequences for gastrins include gastrin 71 of SEQ ID NO. 5, gastrin 52 of SEQ ID NO. 6, gastrin 34 (big gastrin) of SEQ ID NO. 1 or 2, gastrin 17 (little gastrin) of SEQ ID NO. 3 or 4, gastrin 14 of SEQ ID NO. 7, and gastrin 6 of SEQ ID NO. 8 or 9. Gastrin-34 is essentially an extension of an amino acid sequence at the N-terminal end of gastrin-17. Big gastrin is cleaved in vivo to release gastrin-17. Glp at the N-terminal end of a gastrin is pyroglutamate, which is a naturally cyclized form of glutamate. In various embodiments, where cysteine or lysine is added to a terminus of gastrin having a pyroglutamate, the pyroglutamate is replaced with a glutamate, or the pyroglutamate is deleted. A gastrin 34 or gastrin 17 may be used in the invention where there is a methionine or a leucine at position 15, as shown in SEQ ID NOs: 1-4 herein.

Examples of gastrin compounds that may be used in the present invention include the compounds disclosed in U.S. Pat. No. 6,288,301. In some applications of the invention, a gastrin compound may be selected that is a peptide or non-peptide agonist or partial agonist of the gastrin receptor such as A71378 (Lin et al., Am. J. Physiol. 258 (4 Pt 1): G648, 1990).

In some applications of the invention, a gastrin compound may be selected that is a gastrin/CCK receptor ligand including but not limited to cholecystokinin (CCK) such as CCK 58, CCK 33, CCK 22, CCK 12 and CCK 8; and the like. In general, gastrin/CCK receptor ligands share a carboxy terminal sequence Trp-Met-Asp-Phe-amide.

A “gastrin compound” includes a modified form of a gastrin, including but not limited to a modified form of gastrin 71 [SEQ ID NO. 5], gastrin 52 [SEQ ID NO. 6], gastrin 34 (big gastrin) [SEQ ID NO. 1 or 2], gastrin 17 (little gastrin) [SEQ ID NO. 3 or 14], gastrin 14 [SEQ ID NO. 7], gastrin 8, gastrin 6 [SEQ ID NO. 8], pentagastrin, and tetragastrin. A modified gastrin preferably comprises TrpMetAspPhe-NH₂ [SEQ ID NO. 13] or TrpLeuAspPhe-NH₂ [SEQ ID NO.14].

In aspects of the invention a modified gastrin comprises at least amino acids 1-34, 18-34 or 29-34 of SEQ ID NO. 1 or 2, or amino acids 1-17, 2-17, 12-17, or 14-17 of SEQ ID NO. 3 or 4.

A gastrin compound used in aspects of the methods, compositions, and conjugates of the invention may comprise gastrin 17 and analogs and derivatives thereof. In particular aspects, the gastrin compound is synthetic human gastrin 1 having 17 amino acid residues with a Leu residue at amino acid position 15 [SEQ ID NO. 4].

A gastrin compound used in the methods, compositions and conjugates of the invention may comprise gastrin 34 and analogs and derivatives thereof. In particular aspects, the gastrin compound is a synthetic human gastrin 34 with methionine or leucine at position 32 [SEQ ID NO. 1 or 2].

Modified gastrin compounds for use in the present invention comprise the modified gastrin compounds described in PCT/CA03/01778, U.S. Ser. No. 10/719,450 and U.S. Application Ser. No. 60/519,933.

In particular, a modified gastrin can be a gastrin derivative or analogue comprising a minimal sequence of 6 amino acids (from the C-terminal end) of a gastrin, in particular amino acid residues 1 to 34, 18 to 34 or 29-34 of SEQ ID NO: 1 or 2, or amino acid residues 1-17, 2-17, 12-17, or 14-17 of SEQ ID NO. 3 or 4, and comprising a reactive group capable of undergoing an addition reaction. Examples of reactive groups include without limitation thiols, alpha amino groups, epsilon amino groups, carboxyl groups or aromatic rings. A reactive group is generally capable of linking a gastrin sequence, directly or indirectly via a crosslinking agent and/or spacer region, to a carrier.

A reactive group may be introduced by adding or substituting an amino acid comprising a reactive group, for example by adding a cysteine or lysine. Therefore, a modified gastrin may comprise a gastrin sequence (e.g. gastrin-34 or gastrin 17) wherein at least one reactive amino acid (e.g. cysteine or lysine) is added or substituted. The addition of a reactive amino acid can be at a terminal region, in particular an N-terminal region.

A modified gastrin may also optionally comprise a spacer. A spacer can interact with a reactive group, for example, an amino acid comprising a reactive group. A spacer can be one or more amino acids, peptides, peptidomimetics, or small organic molecules. A spacer can comprise at least one amino acid, preferably at least two, three, four or five amino acids and in certain embodiments it is a sequence of several amino acids, including without limitation alanine or glycine. A spacer can comprise alternating amino acids (e.g. glycine and/or alanine), non-alternating amino acids, a random sequence or a particular sequence. By way of example, a spacer can be synthesized as part of, or may be chemically attached to an amino acid of a gastrin sequence.

A modified gastrin may optionally comprise a cross-linking agent. A cross-linking agent may comprise a homobifunctional or heterobifunctional portion for interaction directly or indirectly with a gastrin, spacer and/or a reactive group. A cross-linking agent may interact with a gastrin sequence or a spacer, or it may be added to a reactive group at the end (in particular N-terminus) of a modified gastrin.

A cross-linking agent can be any agent that can link a gastrin sequence and a carrier directly or via a spacer. Examples of homobifunctional crosslinking agents include without limitation amino group directed homobifunctional cross-linking reagents such as bisimidates (e.g. methyl acetimidate-HCl), bifunctional aryl halides (e.g. 1,5-dichloro-2,4-dinitrobenzene), bifunctional acylating agents (e.g. diisocyanates), bifunctional sulfonyl halides (e.g. phenol-2,4-disulfonyl-chloride), bifunctional acylazides (e.g. tartryl diazide), dialdehydes (e.g. glutaraldehyde), and diketones (e.g. 2,5-hexanedione). Examples of heterobifunctional crosslinkers include amino and sulfhydryl group directed bifunctional reagents (e.g. N-succinimidyl-3-(2-pyridyldithio propionate, carboxyl and either sulfhydryl or amino group directed bifunctional reagents (e.g. p-nitrophenyl diazoacetate), and carbonyl and sulfhydryl group directed bifunctional reagents (e.g. 1-(aminooxy)-4-[3-nitro-2-pyridyl)dithio)]butane).

A modified gastrin can optionally comprise a carrier which may be a polymer. A carrier may be a polymer of amino acids (proteins), sugars (polysaccharides), nucleosides, synthetic polymers and mixtures thereof. A protein carrier may be a protein found in the circulatory system. Examples of protein carriers found in the circulatory system, in particular the human circulatory system, include without limitation plasma components such as serum, purified serum proteins such as albumin (in particular human serum albumin), transferrin, or an immunoglobulin, red blood cell proteins such as glycophorin A and AE-1, sugar binding proteins such as a lectin, inactivated enzymes, phosphate and sulphate binding proteins, and lipid binding proteins. Examples of other suitable polymeric carriers include without limitation cellulose and derivatives thereof, starch and derivatives thereof, heparin and derivatives thereof, and synthetic polymers such as polyethylene glycol (PEG) and dextran, and derivatives thereof. Carriers may be attached to a gastrin or spacer by way of reactive groups on, or introduced to, the carrier, gastrin, and/or spacer. For example, carriers can be covalently attached to reactive groups (such as thiol groups, alpha and epsilon amino groups, carboxyl groups or aromatic groups) on a gastrin or spacer which may be present or added by chemical modification of the gastrin or spacer.

In certain aspects of the invention, a modified gastrin can comprise a gastrin of SEQ ID NOS 1, 2, 3, 4, 7, or 8 and a carrier.

A group of modified gastrin compounds include compounds having an amino acid sequence comprising from the amino terminus Z-Yₘ-Xₙ-AA₁-AA₂-AA₃-AA₄-AA₅-AA₆ wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr and wherein AA₆ is optionally amidated; Z is a carrier, in particular a polymer and when the polymer is a protein Z is an amino acid sequence; Yₘ is an optional spacer region comprising m amino acid residues of a small neutral amino acid including but not limited to serine and alanine, and X is any consecutive portion of residues 1-28 (=n) of SEQ ID NO: 1 or 2 or 1-11 of SEQ ID. NO. 3 or 4, providing that the gastrin compound binds a gastrin/CCK_{B} receptor. Generally, m is 0 to about 20 residues. In an aspect Z is a protein, in particular a protein of the circulatory system, more particularly a serum protein, still more particularly albumin, most particularly human serum albumin.

In embodiments, X is one or more amino acid residues from position 18 to position 28 of SEQ ID NO: 1. Therefore, the gastrin compounds by virtue of the presence of X, can have any of gastrin sequences from positions 18-28, 19-28, 20-28, 21-28, etc. The gastrin compound optionally contains an amino acid spacer (Y) of length m, and m is 0 to about 20 residues.

In embodiments, X is one or more amino acid residues from position 1 to 11 or 2 to 11 of SEQ ID NO: 3 or 4. Therefore, the gastrin compounds by virtue of the presence of X, can have any of gastrin sequences from positions 2 to 11, 3 to 11, 4 to 11, 5 to 11, etc. The gastrin compound optionally contains an amino acid spacer (Y) of length m, and m is 0 to about 20 residues.

A gastrin compound includes a modified gastrin compound of the formula Xₙ-AA₁-AA₂-AA₃-AA₄-AA₅-AA₆, where there is no spacer (Y) and m is 0, which may further comprise a bifunctional cross-linking agent for interaction or linkage to a carrier Z, where Z further comprises a non-proteinaceous polymer such as dextran or PEG.

A modified gastrin compound particularly described herein may further comprise an amino terminal cysteine or lysine residue.

In some embodiments of modified gastrin compounds described herein, the gastrin component contains at least amino acid residues 29-34 of SEQ ID NO: 1 or 2, and it is associated with a polymer, a lipid or a carbohydrate. The polymer may be a synthetic or naturally occurring polymer. The term polymer includes a protein polymer of amino acids, and is not limited to a synthetic polymer. The polymer may be a polyethylene glycol (PEG) or a dextran. A modified gastrin compound can be based on SEQ ID NO: 1 or 2 or “big” gastrin-34 and have a residue at position 32 which is a methionine or a leucine, respectively.

Another preferred modified gastrin compound comprises a structure C-Yₘ-X, wherein C is Cys or Lys, Yₘ is an optional spacer region comprising m amino acid residues of a small neutral amino acid, and X is at least six amino acid residues comprising at least positions 12-17 of gastrin-17 (SEQ ID NO: 3 or 4) or at least positions 29-34 of gastrin-34 (SEQ ID NO: 1 or 2). This modified gastrin compound can further comprise a bifunctional cross-linking agent wherein one reactive portion of the cross-linking agent is covalently linked to C, and the other reactive portion is covalently linked to a polymer or protein.

In a particular aspect of the invention AA₁-AA₂-AA₃-AA₄-AA₅-AA₆ in a modified gastrin compound is Tyr-Gly-Trp-Met-Asp-Phe [SEQ ID NO. 10] or Tyr-Gly-Trp-Leu-Asp-Phe [SEQ ID NO. 11].

In a further aspect of the invention, a gastrin compound used in the methods, compositions and conjugates of the invention is gastrin 34 or gastrin 17 or portions thereof, directly or indirectly interacting or associated with a serum protein, in particular albumin or an immunoglobulin, more particularly human serum albumin.

In aspects of the invention, a gastrin compound comprises synthetic human gastrin 34 having 2-34 amino acid residues of SEQ ID NO. 1 or 2, and optionally an N-terminal cysteine and/or a carrier; synthetic human gastrin having 1-17 amino acid residues with a Leu residue at amino acid position 15 [SEQ ID NO. 4] and optionally an N-terminal cysteine residue; and a synthetic human gastrin having amino acid residues 2 to 17 or 5-17 of SEQ ID NO. 3 or 4, optionally with an N-terminal cysteine residue and/or a carrier (e.g. PEG or human serum albumin) linked via a spacer [e.g. Gly-Ala-Gly-Ala-Gly-Ala-Gly-Ala-Gly-Ala i.e. (GA)₅] [SEQ ID NO. 12], in particular, a synthetic human gastrin having amino acid residues 2 to 17 or 5-17 of SEQ ID NO. 3 or 4, with a human serum albumin (HSA) polymer linked via a Gly-Ala-Gly-Ala-Gly-Ala-Gly-Ala-Gly-Ala [i.e. (GA)₅] spacer, and optionally an N-terminal cysteine residue.

In particular aspects of the invention the gastrin compound is a leucine substituted gastrin 17 of SEQ ID NO. 3. Such a gastrin compound may also be characterized by the following properties: isoelectric point of about 3.4; purity of at least about 80%, 85%, 90%, 92%, 93%, 94%, 95%, 96%, 97%, 98%, or 99%, and/or a molecular mass of about 2080.2±2 Da.

In some aspects of the invention a “gastrin compound” is a gastrin agonist. A “gastrin agonist” refers to any substance that fully or partially mimics a reaction, activity, or function of a gastrin compound or initiates such reaction, activity, or function, or reduces or prevents inhibition of any reaction, activity or function of a gastrin compound.

A gastrin agonist may be selected for particular applications in the present invention based on its ability to increase plasma gastrin about 2 to 1000 fold, 2 to 500 fold, 2 to 200 fold, 2 to 100 fold, 2 to 150 fold, 2 to 100 fold, 2 to 75 fold, 2 to 50 fold, 2 to 40 fold, 2 to 30 fold, 2 to 20 fold, 10 to 1000 fold, 10 to 500 fold, 10 to 200 fold, 10 to 150 fold, 10 to 100 fold, 10 to 75 fold, 10 to 50 fold, 10 to 40 fold, 10 to 30 fold or 10 to 20 fold.

In certain aspects of the invention, a gastrin agonist is a substance that increases the secretion of endogenous gastrins, cholecystokinins or similarly active peptides from sites of tissue storage. In some aspects of the invention, a gastrin agonist is a gastrin secretagogue. Examples of gastrin agonists include without limitation proton pump inhibitors (e.g., the gastric releasing peptide, omeprazole which inhibits gastric acid secretion), and soya bean trypsin inhibitor which increases CCK stimulation.

In particular aspects of the invention, a gastrin agonist is selected that provides, in combination with a DPP-IV inhibitor therapeutically effective amounts of gastrin in a subject (e.g. a diabetic subject). In embodiments of the invention a gastrin agonist is selected that provides, in combination with a DPP-IV inhibitor, an about 10 to 1000 fold, 10 to 500 fold, 10 to 100 fold, 10 to 50 fold, 5 to 500 fold, 5 to 100 fold, or 5 to 50 fold increase in plasma gastrin. Examples of gastrin agonists are proton pump inhibitors and histamine-2 receptor antagonists.

A “proton pump inhibitor” and “PPI” are used interchangeably herein and include a substance which inhibits gastric acid secretion by blocking the proton pump, (e.g. a substance which binds covalently to the H+/K+-ATPase, the enzyme responsible for gastric acid secretion) and/or increases gastrin secretion. In particular, the terms refer to any acid labile pharmaceutical agent possessing pharmacological activity as an inhibitor of H⁺/K⁺-ATPase. More particularly it contemplates substances which covalently bind to H+/K+-ATPase. [See the following publications relating to PPIs: Fellenius et al., Substituted Benzimidazoles Inhibit Gastric Acid Secretion by Blocking H⁺, K⁺-ATPase, Nature, 290:159-161 (1981); Wallmark et al, The Relationship Between Gastric Acid Secretion and Gastric H⁺, K⁺-ATPase Activity, J. Biol. Chem., 260:13681-13684 (1985); Fryklund et al., Function and Structure of Parietal Cells After H⁺, K⁺-ATPase Blockade, Am. J. Physiol., 254 (3 PT 1); G399-407 (1988)].

In aspects of the invention a proton pump inhibitor includes compounds comprising a 2-[(2-pyridinyl)methylsulphinyl]-1H-benzimidazole skeleton or related skeletons, where these skeletons may be optionally substituted in various different ways. A proton pump inhibitor may, if desired, be in the form of free base, free acid, salt, ester, solvates (in particular hydrates), anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active.

The following compounds may be mentioned in the context of the present invention: 2-[2-(N-isobutyl-N-methylamino)benzyl-sulphinyl]benzimidazole (INN: leminoprazole) (DE-A-3531487); 2-(4-methoxy-6,7,8,9-tetrahydro-5H-cyclohepta[b]pyridin-9-ylsulphinyl)-1H-benzimidazole (INN: nepaprazole) (EP-A-0 434 999); 2-(4-methoxy-3-methyl-pyridin-2-ylmethylsulphinyl)-5-pyrrol-1-y-1H-benzimidazole (IY-81149), 5-methoxy-2-[(4-methoxy-3,5-dimethyl-2-pyridinyl)methylsulphinyl]-1-H-imidazo[4,5-b]pyridine(tenatoprazole), especially 5-methoxy-2-[(4-methoxy-3,5-dimethyl-2-pyridinyl)methylsulphinyl]-1H-benzimidazole (INN: omeprazole) (U.S. Pat. No. 4,786,505 and EP-A-0 005 129); 5-methoxy-2-[(S)-[(4-methoxy-3,5-dimethyl-2-pyridinyl)methyl]sulphinyl]-1H-benzimidazole (INN: esomeprazole), 2-[3-methyl-4-(2,2,2-trifluoroethoxy)-2-pyridinyl)methylsulphinyl]-1H-benzimidazole (INN: lansoprazole) (EP-A 0 174 726); and 2-{[4-(3-methoxypropoxy)-3-methylpyridin-2-yl]-methylsulphinyl}-1H-benzimidazole (INN: rabeprazole) (EP-A 0 184 322, EP-A 0 254 588, EP-A-0 261 478, EP-A-0 268 956); and in particular 5-difluoromethoxy-2-[(3,4-dimethoxy-2-pyridinyl)methylsulphinyl]-1H-benzimidazole (INN: pantoprazole) (EP-A-0 124 495, EP-A-0 166 287); and (−)-5-difluoromethoxy-2-[(3,4-dimethoxy-2-pyridinyl)methylsulphinyl]-1H-benzimidazole [(−)pantoprazole].

Other proton pump inhibitors include but are not limited to: soraprazan (Altana); ilaprazole (U.S. Pat. No. 5,703,097) (Il-Yang); AZD-0865 (AstraZeneca); hydroxyomeprazole; dontoprazole; habeprazole; perprazole; ransoprazole; pariprazole; YH-1885 (PCT Publication WO 96/05177) (SB-641257) (2-pyrimidinamine, 4-(3,4-dihydro-1-methyl-2(1H)-isoquinolinyl)-N-(4-fluorophenyl)-5,6-dimethyl-monohydro-chloride) (YuHan); phenylalkyl-amino derivatives of condensed carbapenem cpds (WO-A-9523149); BY-112 (Altana); SPI447 (Imidazo[1,2-a]thieno(3,2-c)pyridin-3-amine, 5-methyl-2-(2-methyl-3-thienyl) (Shinnippon); 3-hydroxymethyl-2-methyl-9-phenyl-7H-8,9-dihydro-pyrano(2,3-c)-imidazo[1,2-a]pyridine (PCT Publication WO 95/27714) (AstraZeneca); Pharmaprojects No. 4950 (3-hydroxymethyl-2-methyl-9-phenyl-7H-8,9-dihydro-pyrano(2,3-c)-imidazo[1,2-a]pyridine) (AstraZeneca) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (Toa Eiyo); HN-11203 (Nycomed Pharma); OPC-22575; pumilacidin A (BMS); saviprazole (EP 234485) (Aventis); SKandF-95601 (GSK, discontinued); Pharmaprojects No. 2522 (EP 204215) (Pfizer); S-3337 (Aventis); RS-13232A (Roche); AU-1363 (Merck); SKand F-96067 (EP 259174) (Altana); SUN 8176 (Daiichi Phama); Ro-18-5362 (Roche); ufiprazole (EP 74341) (AstraZeneca); Bay-p-1455 (Bayer); BY308; perprazole; [4-(2,2,2-trifluoroethoxy)-3-methyl-2-pyridyl]-methyl]sulfenamide; (Z)-5-methyl-2-[2-(1-naphthyl)ethenyl]-4-piperidinopyridine HCl; 2-(4-cyclohexyloxy-5-methylpyridin-2-yl)-3-(1-naphthyl)-1-propanol; methyl 2-cyano-3-(ethylthio)-3-(methylthio)-2propenoate; 2-((4-methoxy-2-pyridyl)methylsulphinyl)-5-(1,1,2,2-tetrafluoroethoxy)-1H-benzimidazole sodium; 2-[[[4-(2,2,3,3,4,4,4-heptafluoro butoxy)-2-pyridyl]methyl)sulfinyl]-1H-thieno[3,4-d]imidazole; 2-[[[4-(2,2,2-trifluoro ethoxy)-3-methyl-2-pyridyl]methyl]sulfinyl]-1H-benzimidazole; 2-[[[4-(2,2,2-trifluoro ethoxy)-3-methyl-2-pyridyl]methyl]sulfinyl]-1H-benzimidazole; 2-methyl-8-(phenyl methoxy)-imidazo(1,2-A)-pyridine-3-acetonitrile; (2-((2-dimethylaminobenzyl)sulfinyl)-benzimidazole); 4-(N-allyl-N-methyl amino)-1-ethyl-8-((5-fluoro-6-methoxy-2-benzi midazolyl)sulfinylmethyl)-1-ethyl 1,2,3,4-tetrahydroquinolone; 2-[[(2-dimethylamino phenyl)methyl]sulfinyl]-4,7-dimethoxy-1H-benzimidazole; 2-[(2-(2-pyridyl)phenyl)sulfinyl)-1H-benzimidazole; (2-[(2-amino-4-methylbenzyl)sulfinyl]-5-methoxy benzo[d]imidazole; (4(2-methylpyrrol-3-yl)-2-guanid isothiazole); 4-(4-(3-(imidazole) propoxy)phenyl)-2-phenylthiazole; (E)-2-(2-(4-(3-(dipropylamino)butoxy)phenyl)-ethenyl)benzoxazole; (E)-2-(2-(4-(3-(dipropylamino) propoxy)phenyl)ethenyl)-benzothiazole; Benzeneamine, 2-[[(5-methoxy-1H-benzimidazol-2-yl)sulfinyl]methyl)-4-methyl-; 2,3-dihydro-2-methoxycarbonylamino-1,2-benzisothiazol-3-one; 2-(2-ethyl aminophenylmethylsulfinyl)-5,6-dimethoxybenzimidazole; 2-methyl-8-(phenyl methoxy)imidazo[1,2-a]pyridine-3-acetonitrile; 3-amino-2-methyl-8-phenyl methoxy imidazo[1,2-a)-pyrazine HCl; 2-[[(3-chloro-4-morpholino-2-pyridyl)methyl]-sulfinyl)-5-methoxy-(1H)-benzimidazole; [3-butryl-4-(2-methylphenylamino)-8-methoxy-quinoline); 2-indanyl 2-(2-pyridyl)-2-thiocarbamoylacetate HCl; 2,3-dihydro-2-(2-pyridinyl)-thiazolo (3,2-a)-benzimidazole; 3-cyanomethyl-2-methyl-8-(3-methyl-2-butenyloxy)-(1,2-α-imidazo pyridine; zinc L-carnosine; or, a free base, free acid, salt, hydrate, ester, amide, enantiomer, isomer, tautomer, polymorph, prodrug, or derivative of these compounds.

Still other proton pump inhibitors contemplated by the present invention include those described in the following U.S. Pat. Nos. 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994; 6,479,075; 6,559,167 and in the following patent applications and patents below: DE-A-3531487, EP-A-0 005 129, EP-A-0 124 495, EP-A-0 166 287, EP-A0 174 726, EP-A-0 184 322, EP-A-0 254 588, EP-A-0 261 478, EP-A-1 268 956, EPA-0 434 999 and WO-A-9523149.

In aspects of the invention the proton pump inhibitor is selected from the group consisting of 5-methoxy-2-[(4-methoxy-3,5-dimethyl-2-pyridinyl)-methylsulphinyl]-1H-benzimidazole (omeprazole), 5-methoxy-2-[(S)-[(4-methoxy-3,5-dimethyl-2-pyridi-nyl)methyl]sulphinyl]-1H-benzimidazole(esomeprazole), 2-[3-methyl-4-(2,2,2-trifluoroethoxy)-2-pyridinyl)methylsulphinyl]-1H-benzimidizole(lansoprazole), 2-{[4-(3-methoxypropoxy)-3-methylpyridin-2-yl]methylsulphinyl}-1H-benzimidazole(rabeprazole), 5-difluoromethoxy-2-[(3,4-di-methoxy-2-pyridinyl)-methylsulphinyl]-1H-benzimidazole (pantoprazole) and the hydrates, solvates, salts, hydrates of the salts and solvates of the salts thereof.

In other aspects of the invention a proton pump inhibitor is in the form of a salt. A salt of a proton pump inhibitor may be prepared, for example, from formic, acetic, propionic, succinic, glycolic, gluconic, lactic, malic, tartaric, citric, ascorbic, glucuronic, maleic, ftimaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

In an embodiment, acid addition salts are prepared from the free base of a proton pump inhibitor using conventional methods involving reaction of the free base with a suitable acid. Suitable acids for preparing acid addition salts include without limitation organic acids, such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, such as hydrochloric acid, hydrobromic acid, sulfuric acid, nitric acid, phosphoric acid, and the like.

In another embodiment, an acid addition salt is converted to a free base by treatment with a suitable base. In a further embodiment, an acid addition salt is a halide salt, which is prepared using hydrochloric or hydrobromic acids. In still another embodiment, the basic salt is an alkali metal salt, such as a sodium salt or copper salt.

Examples of salts of proton pump inhibitors include without limitation: a sodium salt form such as esomeprazole sodium, omeprazole sodium, rabeprazole sodium, pantoprazole sodium; or a magnesium salt form such as esomeprazole magnesium or omeprazole magnesium described in U.S. Pat. No. 5,900,424; a calcium salt form; or a potassium salt form such as the potassium salt of esomeprazole described in U.S. patent Ser. No. 02/0198239 and U.S. Pat. No. 6,511,996. Other salts of esomeprazole are described in U.S. Pat. Nos. 4,738,974 and 6,369,085. Salt forms of pantoprazole and lansoprazole are disclosed in U.S. Pat. Nos. 4,758,579 and 4,628,098, respectively.

In an embodiment, esters of proton pump inhibitors are utilized. An ester may be prepared by functionalization of hydroxyl and/or carboxyl groups which may be present within the molecular structure of the drug. In another embodiment, the esters are acyl-substituted derivatives of free alcohol groups, such as moieties derived from carboxylic acids of the formula —RCOOR¹ where R¹ is an alkyl group in particular a lower alkyl group. An ester can be converted to a free acid, if desired, by using conventional procedures such as hydrogenolysis or hydrolysis.

A proton pump inhibitor or its salts can be in a crystalline form. Crystals of a proton pump inhibitor may contain variable amounts of solvent. Therefore, the term “proton pump inhibitor” includes all solvates, in particular all hydrates, of the proton pump inhibitors and their salts.

In particular aspects of the invention the proton pump inhibitor is a salt or hydrate including without limitation pantoprazole-sodium sesquihydrate [pantoprazole-sodium×1.5H₂O], (−)-pantoprazole-sodium sesquihydrate, pantoprazole-magnesium dihydrate, omeprazole-magnesium, omeprazole-magnesium tetrahydrate, esomeprazole-magnesium and esomeprazole-magnesium tetrahydrate.

In various aspects of the invention, the proton pump inhibitor is a substituted bicyclic aryl-imidazole, wherein the aryl group may be, for example, a pyridine, a phenyl, or a pyrimidine group which is attached to the 4- and 5-positions of the imidazole ring. Proton pump inhibitors comprising a substituted bicyclic aryl-imidazole include, but are not limited to, omeprazole, hydroxyomeprazole, esomeprazole, lansoprazole, pantoprazole, rabeprazole, dontoprazole, habeprazole, perprazole, tenatoprazole, ransoprazole, pariprazole, leminoprazole, or a free base, free acid, salt, hydrate, ester, amide, enantiomer, isomer, tautomer, polymorph, prodrug, or derivative thereof. (See, e.g., The Merck Index, Merck & Co. Rahway, N.J. (2001)).

Substituted bicyclic aryl-imidazole compounds as well as their salts, hydrates, esters, amides, enantiomers, isomers, tautomers, polymorphs, prodrugs, and derivatives may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry, (1992); Howarth et al.; Core Organic Chemistry (1998); and Weisermel et al., Industrial Organic Chemistry (2002).

A tautomer of a substituted bicyclic aryl-imidazole includes without limitation tautomers of omeprazole such as those disclosed in U.S. Pat. Nos. 6,262,085; 6,262,086; 6,268,385; 6,312,723; 6,316,020; 6,326,384; 6,369,087; and 6,444,689; and U.S. Publication No. 02/0156103. An example of an isomer of a substituted bicyclic aryl-imidazole is an isomer of omeprazole including but not limited to an isomer disclosed in: Oishi et al., Acta Cryst. (1989), C45, 1921-1923; U.S. Pat. No. 6,150,380; U.S. patent publication Ser. No. 02/0156284; and PCT Publication No. WO 02/085889.

An amide of a bicyclic aryl-imidazole compound may be prepared using techniques known to those skilled in the art or described in the pertinent literature. For example, an amide may be prepared from esters, using suitable amine reactants, or they may be prepared from an anhydride or an acid chloride by reaction with an amine group e.g., ammonia or a lower alkyl amine.

Suitable polymorphs include but are not limited to the polymorphs described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5; 5,731,006; 5,879,708; 5,900,424; 5,948,773; 5,948,789; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,384,059, 6,428,810; 6,444,689; 6,462,058; 6,903,122; 6,933,389; and 6,939,971.

In aspects of the invention, a proton pump inhibitor suitable for use in the invention is a benzimidazole compound, for example, a benzimidazole compound described in the following patent documents U.S. Pat. Nos. 4,045,563; 4,255,431; 4,359,465; 4,472,409; 4,508,905; 4,628,098; 4,738,975; 5,045,321; 4,786,505; 4,853,230; 5,045,552, and 5,312,824; EP-A-295603; EP-A-166287; EP-A-519365; EP5129; EP 174,726; EP 166,287; GB 2,163,747; and JP-A-59181277.

In embodiments of the invention the proton pump inhibitor is leminoprazole, nepaprazole, tenatoprazole, omeprazole, esomeprazole, lansoprazole, rabeprazole, pantoprazole, pariprazole, (−)pantoprazole, soraprazan, ilaprazole, AZD-0865, hydroxyomeprazole, dontoprazole, habeprazole, periprazole, ransoprazole, pariprazole, or a free base, free acid, salt, hydrate, ester, amide, enantiomer, isomer, tautomer, polymorph, metabolite or prodrug thereof.

In particular embodiments of the invention, the proton pump inhibitor is one or more of omeprazole, hydroxyomeprazole, esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, metabolite or prodrug thereof.

In other particular embodiments of the invention, the proton pump inhibitor is one or more of tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, metabolite or prodrug thereof.

In particular aspects of the invention a proton pump inhibitor comprises or is selected from the group consisting of omeprazole, hydroxyomeprazole, esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, or prodrug thereof.

Preferred proton pump inhibitors are esomeprazole (Nexium), lansoprazole (Zoton), pantoprazole (Protium), rabeprazole sodium (Pariet), or a pharmaceutically acceptable salt or isomer thereof.

A “histamine-2 receptor antagonist” or “H-2 antagonist” refers to a compound which blocks H-2 receptors, but does not have meaningful activity in blocking histamine-1 receptors. Selective H-2 antagonists include compounds which are disclosed in U.S. Pat. Nos. 5,294,433, 5,364,616, and US Patent Application No. 20050042283, including without limitation cimetidine [Merck Index, 11th edition (1989), p. 354 (entry no. 2279) and Physicians' Desk Reference, 46th edition (1992), p. 2228]; etintidine (U.S. Pat. No. 4,112,234); ranitidine or its hydrochloride salt (AH-19065) [U.S. Pat. No. 4,128,658, Merck Index, 11th edition (1989), p. 1291 (entry no. 8126), and Physicians' Desk Reference, 46th edition (1992), p. 1063]; hydroxymethyl ranitidine; ranitidine bismuth citrate (GR-122311, GR-122311X); AH-18801; N-cyano-N′-(2-(((5-((dimethylamino)methyl)-2-f-uranyl)methyl)thio)ethyl)-N″-methyl-guanidine; tiotidine (U.S. Pat. No. 4,165,378); ORF-17578 (U.S. Pat. No. 4,203,909); lupitidine (SKF-93479) (U.S. Pat. No. 4,234,588); donetidine (SKF-3574); famotidine (YM-11170, MK-208) [Merck Index, 11th edition (1989), p. 617 (entry no. 3881), and Physicians' Desk Reference, 46th edition (1992), p. 1524]; roxatidine or rozatidine acetate [U.S. Pat. No. 4,293,557, Merck Index, 11^{th} edition (1989), p. 1316 (entry no. 8252)]; pifatidine; lamtidine (U.S. Pat. No. 4,318,913); BL-6548; BMY-25271; zaltidine (U.S. Pat. No. 4,374,843); nizatidine [U.S. Pat. No. 4,375,547, Merck Index, 11^{th} edition (1989), p. 1052 (entry no. 6582), and Physicians' Desk Reference, 46^{th} edition (1992), p. 1246)]; mifentidine and its hydrochloride salt [(U.S. Pat. No. 4,386,099, Merck Index, 11^{th} edition (1989), p. 973 (entry no. 6108)]; ICIA-5165 (U.S. Pat. No. 4,165,377); BMY-25368 (SKF-94482) (U.S. Pat. No. 4,390,701); SYF-94482; ICI-162846 (U.S. Pat. No. 4,451,463); ramixotidine (U.S. Pat. No. 4,474,790); BL-6341A (BMY-26539) (U.S. Pat. No. 4,394,508); Wy-45727 (U.S. Pat. No. 4,490,527); SR-58042 (U.S. Pat. No. 4,514,408); BMY-25405 (U.S. Pat. Nos. 4,528,377 and 4,600,779); loxtidine (U.S. Pat. No. 4,536,508); DA-4634 (U.S. Pat. Nos. 4,548,944 and 4,645,841); bisfentidine (U.S. Pat. No. 4,649,150); sufotidine (U.S. Pat. No. 4,670,448); ebrotidine (U.S. Pat. No. 4,728,755) HE-30-256 (U.S. Pat. No. 4,738,960); D-16637 (U.S. Pat. No. 4,738,983); FRG-8813 (U.S. Pat. Nos. 4,912,101 and 4,977,267); FRG-8701 (U.S. Pat. No. 4,837,316); impromidine (U.K. Patent Specification No. 1,531,237); L-643728 (European Patent Application No. 0,040,696); MK-208 (U.S. Pat. No. 4,283,408); HB408 (European Patent Application No. 0,186,275); burimamide; and, metiamide.

A “peroxisome proliferator-activated receptor compound” or (PPAR compound) refers to a peroxisome proliferator-activated receptor, in particular peroxisome proliferator-activated receptor α or γ, or agonist thereof, including a PPAR ligand, in particular synthetic PPAR ligands such as fibrate compounds or thiazolidinediones. Examples of PPAR compounds are fenofibrate, micronized fenofibrate, bezafibrate, gemfibrazil and ciprofibrate, or the pharmaceutically acceptable salts of such a compound. PPAR compounds are disclosed in The Merck Index, 13th Edition, (2001), the contents of which is hereby incorporated by reference in its entirety as if set forth in full herein.

“Condition(s) and/or disease(s)” refers to one or more pathological symptoms or syndromes for which either or both a DPP-IV inhibitor or a gastrin compound provide a beneficial or therapeutic effect. The condition and/or disease may require reduction of blood glucose levels, inhibition of gastric acid secretion, inhibition of apoptosis of β-cells, stimulation of proliferation or differentiation of β-cells, and reduction of body weight. Examples of conditions and/or diseases include but are not limited to dyslipidemia, hyperglycemia, severe hypoglycemic episodes, stroke, left ventricular hypertrophy, arrhythmia, bacteraemia, septicaemia, irritable bowel syndrome, functional dyspepsia, diabetes, catabolic changes after surgery, stress induced hyperglycemia, respiratory distress syndrome, gastric ulcers, myocardial infarction, impaired glucose tolerance, hypertension, chronic heart failure, fluid retentive states, metabolic syndrome and related diseases and disorders, obesity, diabetic complications as well as symptoms of other diseases in which tissue is damaged due to elevated glucose levels, including Alzheimer's Disease, Parkinson's Disease, and other age-related, tissue-degenerative diseases, as well as the artherogenic effects of elevated leptin, for example in patients with impaired glucose tolerance and obese non-diabetic patients.

In aspects of the invention the condition and/or disease is diabetes, in particular Type II diabetes mellitus, conditions of impaired glucose tolerance (IGT), conditions of impaired fasting plasma glucose, metabolic acidosis, ketosis, arthritis, obesity and osteoporosis, inflammatory bowel disease, Colitis Ulcerosa, Morbus Crohn, and/or metabolic syndrome or B-cell protection. Preferably the compositions, conjugates and methods of the invention are utilized for the treatment and/or prophylaxis of non-insulin dependent diabetes mellitus and/or impaired glucose tolerance.

The term, “diabetes” as used herein means any manifested symptoms of diabetes in any mammal including experimental animal models, and including human forms such as Type I and Type II diabetes, early stage diabetes, and a pre-diabetic condition characterized by mildly decreased insulin or mildly elevated blood glucose levels. Diabetes disease processes may be derived from multiple causative factors and are characterized by elevated levels of plasma glucose or hyperglycemia in the fasting state or after administration of glucose during an oral glucose tolerance test. Increased and premature morbidity and mortality are associated with persistent or uncontrolled hyperglycemia. Abnormal glucose homeostasis may be associated both directly and indirectly with alterations of lipid, lipoprotein and apolipoprotein metabolism and other metabolic and hemodynamic diseases. Patients with Type II diabetes mellitus therefore may be at an increased risk of macrovascular and microvascular complications, including coronary heart disease, stroke, peripheral vascular disease, hypertension, nephropathy, neuropathy, and retinopathy.

A “pre-diabetic condition” describes a subject demonstrating a symptom in terms of insulin or glucose level, and/or demonstrating a susceptibility to diabetes or a related condition due to family history, genetic predisposition, or obesity in the case of Type II diabetes, and includes a subject who has previously had diabetes or a related condition and is subject to risk of recurrence.

The conditions and/or diseases associated with diabetes, particularly Type II diabetes mellitus, include but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

In aspects of the invention, a condition and/or disease may be selected from the group consisting of (a) Type I or Type II diabetes mellitus and related diseases, disorders or conditions (including but not limited to diabetic nephropathy, diabetic retinopathy and diabetic neuropathy); (b) insulin resistance and syndrome X, obesity and related diseases, disorders or conditions (including but not limited to Insulin Resistance, Type II Diabetes Mellitus, Reproductive Disorders, Cardiovascular Disease, Pulmonary Disease, Gallstones and Fasting-induced cholecystitis, Cancers and Cutaneous Disease), Cushing's Syndrome, Hypothyroidism, Insulinoma, Craniopharyngioma and Other Disorders Involving the Hypothalamus; (c) congestive heart failure, left ventricular hypertrophy, survival post myocardial infarction (MI), coronary artery diseases, atherosclerosis, angina pectoris, thrombosis, (d) hypertension including hypertension in the elderly, familial dyslipidemichypertension and isolated systolic hypertension (ISH); increased collagen formation, fibrosis, and remodeling following hypertension (antiproliferative effect of the combination); impaired vascular compliance, stroke; all these diseases or conditions associated with or without hypertension, (e) renal failure, in particular chronic renal failure, glomerulosclerosis, nephropathy; (f) hypothyroidism; (g) endothelial dysfunction with or without hypertension, (h) hyperlipidemia, hyperlipoproteinemia, hypertryglyceridemia, and hypercholesterolemia, (i) macular degeneration, cataract, glaucoma, j) skin and connective tissue disorders, and (k) restenosis after percutaneous transluminal angioplasty, and restenosis after coronary artery bypass surgery; peripheral vascular disease

“Insulinotropic activity” refers to an ability of a substance to stimulate insulin secretion in response to elevated glucose levels to produce or increase glucose uptake by cells and decreased serum glucose or blood glucose levels. Methods known in the art can be employed to assay for insulinotropic activity. For example, in vitro and in vivo methods may be used that measure insulin or C-peptide levels. Compounds, compositions or conjugates described herein have insulinotropic activity if islet cells secrete insulin in the presence of the compounds, compositions, or conjugates above background levels or levels in the absence of the compounds, compositions, or conjugates. A compound may be administered to an animal and the insulin concentration can be monitored over time.

“Islet neogenesis” means formation of new beta cells by differentiation, which may or may not have the characteristics of stem cells which have the ability to reproduce in an unlimited manner.

### DETAILED DESCRIPTION OF EMBODIMENTS OF THE INVENTION

The invention is related to compositions, conjugates, and methods that utilize a DPP-IV inhibitor and a gastrin compound. In particular, the invention relates to compositions, conjugates, and methods for the prevention, intervention and/or treatment of a condition and/or disease discussed herein comprising at least one DPP-IV inhibitor and at least one gastrin compound. In aspects of the invention, the compositions, conjugates and methods of the invention provide beneficial effects, in particular enhanced beneficial effects, more particularly sustained beneficial effects relative to a DPP-IV inhibitor and/or a gastrin compound alone. The beneficial effects may be complementary, additive or synergistic effects.

In aspects of the invention, where the condition and/or disease is diabetes, beneficial effects, in particular sustained beneficial effects of a composition, combination treatment, or conjugate of the invention may manifest as one or more of the following:
a) An increase in pancreatic insulin levels relative to the levels measured in the absence of the active compounds or for each compound alone after administration to a subject with symptoms of diabetes. Preferably the compounds together induce at least about a 0.05%, 0.1%, 0.5%, 1%, 2%, 5%, 10%, 15%, 20%, 30%, 33%, 35%, 40%, 45%, or 50% increase in pancreatic insulin levels in a subject.
b) A reduction of an absence of symptoms of islet inflammation after administration to a subject with symptoms of diabetes.
c) A decrease in blood glucose levels relative to the levels measured in the absence of the compounds or for each compound alone in subjects with symptoms of diabetes. Preferably, the compounds induce at least about a 1%, 2%, 5%, 10%, 15%, 20%, 30%, 40%, 50%, 60%, 70%, 80%, or 90% decrease in blood glucose levels. Most preferably, the compounds yield blood glucose levels about or close to the levels common in a normal subject.
d) An increase in C-peptide levels relative to the levels measured in the absence of the compounds or for each compound alone in subjects with symptoms of diabetes. Preferably, the compounds together induce at least about a 0.05%, 0.1%, 0.5%, 1%, 2%, 5%, 10%, 15%, 20%, 30%, 33%, 35%, 40%, 45%, or 50% increase in C-peptide levels.
e) Maintenance of blood glucose levels at about normal, in particular for a prolonged period of time.
f) Maintenance of hemoglobin Alc or glycated hemoglobin at about normal levels for a prolonged period of time, in particular maintaining a % hemoglobin Alc at between 6-8%, more particularly at about 7%.
g) A reduction in destruction of beta-cells. Preferably the compounds induce at least about a 1%, 2%, 5%, 10%, 15%, 20%, 30%, 40%, 50%, 60%, 70%, 80%, or 90% reduction.
h) An increase in beta-cell function. Preferably the compounds induce at least about a 1%, 2%, 5%, 10%, 15%, 20%, 30%, 40%, 50%, 60%, 70%, 80%, or 90% increase in beta-cell function.
i) A decrease in insulin delivery or usage compared with the absence of the compounds or for each compound alone in diabetic subjects. Preferably, the compounds provide at least about a 1%, 2%, 5%, 10%, 15%, 20%, 25%, 30%, 35%, 40%, 45%, 50%, 55%, 60%, 65%, 70%, 75%, 80%, 85%, 90%, 92%, 93%, 94%, 95%, 96%, 97%, 98%, 99%, 100%, 30-100%, 30-80%, or 35-75%, reduction in insulin delivery or usage.
j) A decrease in requirement for insulin injection/intake by at least 5-99%, 5-95%, 10-98%, 10-95%, 10-90%, 10-80%, 10-70%, 10-60%, 10-50%, 10-40%, 10-30%, 10-20%, 20-100%, 20-75%, 30-100% 30-90%, 30-80%, 30-75%, 35-90%, 35-80%, or 35-75%.
k) A reduction, prevention, or slowing of the rate of disease progression in a subject with diabetes.
l) A reduction or prevention of the development of severe hyperglycemia and ketoacidosis with symptoms of diabetes.
m) An increase in survival in a subject with symptoms of diabetes.

In embodiments of the invention, beneficial effects or sustained beneficial effects comprise or consist essentially of two, three, four, five, six, seven, eight, nine, ten, eleven, twelve or thirteen of a) through m). In particular embodiments, beneficial effects or sustained beneficial effects comprise or consist essentially of a), b), and c); a), b), c), and d); a), b), c), d), and e); a), b), c), d), e), and f); a), b), c), d), e), f), and g); a), b), c), d), e), f), g), and h); a), b), c), d), e), f), g), h), and i); a), b), c), d), e), f), g), h), i) and j); a), d), and e); a), d), e), and h); a), d), e), h), and i); a), d), e), h), i), and j); a), b), c), d), e), h), i), and j); a), b), c), d), e), h), i), j), and k); b), c), d), and e); b), c), d), e), h), i), and j); b), h), i) and j); a) through e); a) through f); a) through g); a) through h); a) through i); a) through j); a) through k); a) through l); and a) through m).

One or more of these beneficial effects or sustained beneficial effects can be demonstrated in a diabetic subject or disease model, for example a non-obese (NOD) mouse with symptoms of diabetes, using standard methods known to the skilled artisan. For example, commercially available methods and kits may be used to assay pancreatic insulin levels, glucose levels, C-peptide levels and haemoglobin Alc.

A gastrin compound may be selected for particular embodiments in the present invention and to provide a specific beneficial effect(s) based on characteristics including its insulinotrophic activity, the ability to augment the activity of a DPP-IV inhibitor, and/or increase the physical or chemical stability of a DPP-IV inhibitor. A gastrin compound can also be selected based on its ability to stimulate proliferation/differentiation of beta cells, and its in vivo half-life.

In aspects of the invention a gastrin compound comprises an amino acid sequence comprising, from the amino terminus, Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr; Z is an optional polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 1 or 2, or residues 1-17 of SEQ ID NO. 3 or 4, preferably AA₁-AA₂-AA₃-AA₄-AA₅-AA₆ is Tyr-Gly-Trp-Met-Asp-Phe or Tyr-Gly-Trp-Leu-Asp-Phe

In aspects of the invention, a gastrin compound used in the methods, compositions, and conjugates disclosed herein is gastrin 17 and analogs and derivatives thereof, optionally associated with a polymer. In a particular aspect, the gastrin compound is synthetic human gastrin I having 17 amino acid residues with a Leu residue at amino acid position 15 [SEQ ID NO. 4].

In other aspects of the invention, a gastrin compound used in the methods, compositions and conjugates disclosed herein is gastrin 34 and analogs and derivatives thereof. In a particular aspect, the gastrin compound is a synthetic human gastrin 34 with methionine or leucine at position 32 [SEQ ID NO. 1 or 2].

In further embodiments of the invention, a gastrin compound used in the methods, compositions and conjugates of the invention is gastrin 34 or gastrin 17 or portions thereof, directly or indirectly interacting or associated with a serum protein, in particular albumin or an immunoglobulin, more particularly human serum album.

In aspects of the invention, a gastrin compound comprises synthetic human gastrin 34 having 2-34 amino acid residues of SEQ ID NO. 1 or 2, and optionally an N-terminal cysteine and/or a carrier; synthetic human gastrin having 1-17 amino acid residues with a Leu residue at amino acid position 15 [SEQ ID NO. 4] and optionally an N-terminal cysteine residue; and a synthetic human gastrin having amino acid residues 2 to 17 or 5-17 of SEQ ID NO. 3 or 4, optionally with an N-terminal cysteine residue and/or a carrier (e.g. PEG or human serum albumin) linked via a spacer [e.g. Gly-Ala-Gly-Ala-Gly-Ala-Gly-Ala-Gly-Ala i.e. (GA)₅] [SEQ ID NO. 12], in particular, a synthetic human gastrin having amino acid residues 2 to 17 or 5-17 of SEQ ID NO. 3 or 4, with a human serum albumin (HSA) polymer linked via a Gly-Ala-Gly-Ala-Gly-Ala-Gly-Ala-Gly-Ala [ie. (GA)₅] spacer, and optionally an N-terminal cysteine residue.

A DPP-IV inhibitor may be selected for particular applications in the present invention based on one or more of the following characteristics: extending the action of insulin; suppressing the release of glucagons; increasing beta cell production; and/or and inhibiting programmed cell death (apoptosis).

In aspects of the invention, the DPP-IV inhibitor comprises or is selected from the inhibitors referenced in Table 1.

In particular aspects of the invention the DPP-IV inhibitor is Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

In embodiments of the invention the DPP-IV inhibitor is Sitagliptin, Vildagliptin, or Saxagliptin, in particular Vildagliptin (Galvus®).

Pharmaceutical compositions of the invention can be selected that preferably provide beneficial effects, in particular statistically significant beneficial effects or sustained beneficial effects, compared with a DPP-IV inhibitor or a gastrin compound alone. Preferably, there is at least one beneficial effect, e.g., a mutual enhancing of the effect of a DPP-IV inhibitor in free or pharmaceutically acceptable salt form and at least one gastrin compound, additional advantageous effects, less side effects, a combined therapeutic effect in a non-effective dosage of one or each of the components, and especially a synergistic effect, e.g., a more than additive effect, between a DPP-IV inhibitor in free or pharmaceutically acceptable salt form, and a gastrin compound. Beneficial effects in respect to a diabetic condition may be evidenced by one or more of the beneficial effects described herein, in particular one, two, three, four, five, six, seven, eight, nine, ten or more of the beneficial effects described above in a) through m).

Certain aspects of the invention concern a combination for simultaneous, separate or sequential use. Preferably the combination is a pharmaceutical composition comprising at least one DPP-IV inhibitor in free or pharmaceutically acceptable salt form, and at least one gastrin compound in free or pharmaceutically acceptable salt form. Preferably the pharmaceutical composition is a combined preparation or a fixed combination. Preferably the pharmaceutical composition is a combined preparation for simultaneous, separate or sequential use. A combined preparation is also contemplated which comprises a DPP-IV inhibitor in free or pharmaceutically acceptable salt form and at least one gastrin compound in free or pharmaceutically acceptable salt form and optionally at least one, i.e., one or more, e.g., two, pharmaceutically acceptable carrier for simultaneous, separate or sequential use.

A pharmaceutical composition preferably with beneficial effects, more preferably statistically significant beneficial effects or sustained beneficial effects, is provided comprising a DPP-IV inhibitor preferably selected from the group consisting of one or more DPP-IV inhibitor disclosed in a reference listed in Table 1, in particular Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, and L-allo-isoleucyl pyrrolidine described in U.S. Pat. No. 6,001,155, WO 99/61431, WO 99/67278, WO 99/67279, DE 198 34 591, WO 97/40832, DE 196 16 486 C₂, WO 98/19998, WO 00/07617, WO 99/38501, and WO 99/46272, and US20050222221, and one or more gastrin compound (e.g. SEQ ID NO. 1, 2, 3 or 4).

A pharmaceutical composition in particular with beneficial effects, more particularly statistically significant beneficial effects or sustained beneficial effects, is provided comprising one or more DPP-IV inhibitor selected from the group consisting of a DPP-IV inhibitor disclosed in a reference listed in Table 1, in particular Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, and L-allo-isoleucyl pyrrolidine described in U.S. Pat. No. 6,001,155, WO 99/61431, WO 99/67278, WO 99/67279, DE 198 34 591, WO 97/40832, DE 196 16 486 C₂, WO 98/19998, WO 00/07617, WO 99/38501, and WO 99/46272, and US20050222221, and one or more gastrin compound having an amino acid sequence comprising, from the amino terminus, Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr; Z is an optional polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 1 or 2, or residues 1-11 of SEQ ID NO. 3 or 4, preferably AA₁-AA₂-AA₃-AA₄-AA₅-AA₆ is Tyr-Gly-Trp-Met-Asp-Phe or Tyr-Gly-Trp-Leu-Asp-Phe. In a particular embodiment, Z is a serum protein, in particular human serum albumin.

In an aspect, a pharmaceutical composition with statistically significant beneficial effects or sustained beneficial effects is provided comprising one or more DPP-IV inhibitor and one or more of a gastrin compound of any one of SEQ ID NOs. 1 to 9 or modifications thereof, in particular gastrin-34(leu) [SEQ ID NO. 2] or gastrin-17(leu) [SEQ ID NO. 4].

In an aspect, a pharmaceutical composition with statistically significant beneficial effects or sustained beneficial effects is provided comprising one or more DPP-IV inhibitor selected from the group consisting of Sitagliptin, Vildagliptin, PSN9301, saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, and L-allo-isoleucyl pyrrolidine and one or more of a gastrin compound of any one of SEQ ID NOs. 1 to 9 or modifications thereof, in particular gastrin-34(leu) [SEQ ID NO. 2] or gastrin-17(leu) [SEQ ID NO. 4].

In an aspect, a pharmaceutical composition with statistically significant beneficial effects or sustained beneficial effects is provided comprising one or more DPP-IV inhibitor selected from the group consisting of a DPP-IV inhibitor disclosed in a reference listed in Table 1, especially Sitagliptin, Vildagliptin, PSN9301, saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine- and gastrin-17(leu) [SEQ ID NO.4]

In an aspect, a pharmaceutical composition with statistically significant beneficial effects or sustained beneficial effects is provided comprising one or more DPP-IV inhibitor selected from the group consisting of a DPP-IV inhibitor disclosed in a reference listed in Table 1 especially Sitagliptin, Vildagliptin, PSN9301, saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine and one or more gastrin agonist selected from the group consisting of esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, metabolite or prodrug thereof.

In certain aspects of the invention, pharmaceutically acceptable salts of a DPP-IV inhibitor and/or pharmaceutically acceptable salts of a gastrin compound are utilized.

The invention in particular aspects provides a pharmaceutical composition which has been adapted for administration to a subject to provide sustained beneficial effects to treat a condition and/or disease, in particular diabetes. In an embodiment for the prevention and/or treatment of diabetes, the composition is in a form such that administration to a subject results in blood glucose levels that are about normal, and in particular levels that persist in the subject for a prolonged period of time after cessation of treatment.

This invention provides a conjugate comprising a DPP-IV inhibitor linked to or interacting with a gastrin compound wherein the interaction is for example, via an amino or a carboxyl group. The invention also relates to isolated covalent conjugates of the invention, and compositions comprising covalent conjugates of the invention. A DPP-IV inhibitor may be conjugated to a species via an ester bond between an OH and a COOH. Conjugates of a DPP-IV inhibitor and a gastrin compound may be conjugated with an intermediate spacer or linker. A suitable spacer or linker may be a mono- or disaccharide, an amino acid, a sulfate, a succinate, an acetate, or an oligomeric polymeric spacer or linker comprising one or more of such moieties.

The invention also provides methods of preparing conjugates that result in conjugates with improved pharmacokinetic properties, biological activity, and beneficial effects. The methods comprise incubating the DPP-IV inhibitor with a gastrin compound under conditions that allow formation of a covalent linkage between the compounds. The invention therefore contemplates a process for preparing a covalent conjugate comprising a DPP-IV inhibitor covalently bonded or linked to a gastrin compound, the process comprising: incubating the DPP-IV inhibitor with a gastrin compound under conditions and at a pH and for a time sufficient for formation of a covalent bond or linkage between the DPP-IV inhibitor and gastrin compound; and isolating the covalent conjugate. The above process for preparing a conjugate comprising a DPP-IV inhibitor and a gastrin compound may provide a conjugate with a substantial amount of a DPP-IV inhibitor covalently linked to the DPP-IV inhibitor.

N-terminal or C-terminal fusion proteins or chimeric proteins, comprising a peptide DPP-IV inhibitor conjugated with a gastrin compound, optionally with a spacer or linker, may also be prepared by fusing, through recombinant techniques, the N-terminal or C-terminal sequence of a peptide DPP-IV inhibitor and the sequence of a gastrin compound.

The invention relates to a conjugate prepared by a process described herein. The invention also relates to pharmaceutical formulation or composition comprising conjugates of the invention and a pharmaceutically acceptable carrier, excipient, or vehicle.

The invention further relates to a pharmaceutical formulation or composition of substantially pure covalent conjugates comprising a DPP-IV inhibitor covalently linked to a gastrin compound which provides beneficial effects preferably sustained beneficial effects compared to the DPP-IV inhibitor alone. In an embodiment, a pharmaceutical formulation is provided comprising or consisting essentially of covalent conjugates comprising a DPP-IV inhibitor covalently linked without an intermediate spacer or linker to a gastrin compound. In another embodiment, a pharmaceutical formulation is provided comprising or consisting essentially of covalent conjugates comprising a DPP-IV inhibitor covalently linked with an intermediate spacer or linker to a gastrin compound

In aspects of the invention, a composition or conjugate comprising a DPP-IV inhibitor and a gastrin compound have greater sustained insulinotropic activity following treatment compared with the activity of a DPP-IV inhibitor or gastrin compound alone.

The invention provides methods for the prevention, treatment and/or intervention of a condition and/or disease in a subject comprising administering at least one gastrin compound and at least one DPP-IV inhibitor, or a pharmaceutical composition of the invention to provide a beneficial effect, in particular a sustained beneficial effect.

In aspects of methods of the invention, at least one DPP-IV inhibitor and at least one gastrin compound are administered. In particular aspects, the DPP-IV inhibitor is a DPP-IV inhibitor disclosed in a reference listed in Table 1, in particular Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

In certain methods of the invention, Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine are administered. In embodiments of methods of the invention, Sitagliptin, Vildagliptin, or Saxagliptin, N-(N′-substituted glycyl)-2-cyanopyrrolidines, preferably Vildagliptin, are administered.

In aspects of the invention a gastrin compound is administered comprising an amino acid sequence comprising, from the amino terminus, Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr; Z is an optional polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 1 or 2, or residues 1-17 of SEQ ID NO. 3 or 4, preferably AA₁-AA₂-AA₃-AA₄-AA₅-AA₆ is Tyr-Gly-Trp-Met-Asp-Phe or Tyr-Gly-Trp-Leu-Asp-Phe.

In certain methods of the invention providing beneficial effects, in particular statistically significant beneficial effects or sustained beneficial effects, a DPP-IV inhibitor is selected from the group consisting of a DPP-IV inhibitor disclosed in a reference listed in Table 1, in particular Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine and a gastrin compound comprises an amino acid sequence comprising, from the amino terminus, Z-Yₘ-Xₙ-AA₁-AA₂-AA₃-AA₄-AA₅-AA₆, wherein AA₁ is Tyr or Phe, AA₂ is Gly, Ala, or Ser, AA₃ is Trp, Val, or Ile, AA₄ is Met or Leu, AA₅ is Asp or Glu, and AA₆ is Phe or Tyr; Z is an optional polymer and when the polymer is a protein Z is an amino acid sequence; Yₘ is an optional spacer region comprising m amino acid residues of a small neutral amino acid including but not limited to serine and alanine, and X is any consecutive portion of residues 1-28 of SEQ ID NO: 1 or 2, or residues 1-17 of SEQ ID NO. 3 or 4, preferably AA₁-AA₂-AA₃-AA₄-AA₅-AA₆ is Tyr-Gly-Trp-Met-Asp-Phe or Tyr-Gly-Trp-Leu-Asp-Phe. In a particular embodiment, Z is a serum protein, in particular human serum albumin.

In particular aspects of the invention, a gastrin compound is administered in combination with a DPP-IV inhibitor, wherein the gastrin compound comprises synthetic human gastrin 34 having 2-34 amino acid residues of SEQ ID NO. 1 or 2, and optionally an N-terminal cysteine and/or a carrier; synthetic human gastrin having 1-17 amino acid residues with a Leu residue at amino acid position 15 [SEQ ID NO. 4] and optionally an N-terminal cysteine residue; and a synthetic human gastrin having amino acid residues 2 to 17 or 5-17 of SEQ ID NO. 3 or 4, optionally with an N-terminal cysteine residue and/or a carrier (e.g. PEG or human serum albumin) linked via a spacer [e.g. Gly-Ala-Gly-Ala-Gly-Ala-Gly-Ala-Gly-Ala i.e. (GA)₅] [SEQ ID NO. 12], in particular, a synthetic human gastrin having amino acid residues 2 to 17 or 5-17 of SEQ ID NO. 3 or 4, with a human serum albumin (HSA) polymer linked via a Gly-Ala-Gly-Ala-Gly-Ala-Gly-Ala-Gly-Ala [ie. (GA)₅] spacer, and optionally an N-terminal cysteine residue.

In an embodiment of methods of the invention, a gastrin 17 or analog or derivative thereof, in particular synthetic human gastrin I having 17 amino acid residues with a Leu residue at amino acid position 15 [SEQ ID NO. 4], is administered, in combination with a DPP-IV inhibitor, especially Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

In other embodiments of methods of the invention, a gastrin 34 or analog or derivative thereof, in particular aspect, synthetic human gastrin 34 with methionine or leucine at position 32 [SEQ ID NO. 1 or 2], is administered in combination with a DPP-IV inhibitor, especially Sitagliptin, Vildagliptin, PSN9301, Saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

In certain aspects of methods of the invention, pharmaceutically acceptable salts of a DPP-IV inhibitor and/or gastrin compound are utilized.

The invention provides a method for the prevention and/or intervention of a condition and/or disease discussed herein in a subject comprising administration of at least one DPP-IV inhibitor and at least one gastrin compound. A DPP-IV inhibitor and a gastrin compound may be directly administered to a subject or contacted with cells (e.g. stem cells or progenitor cells) and administered to a subject.

The invention also provides a combination treatment for preventing and/or treating a condition and/or disease discussed herein in a subject comprising administering to the subject a therapeutically effective amount of at least one DPP-IV inhibitor and at least one gastrin compound to provide beneficial effects. In an aspect the invention provides a combination treatment or intervention which provides sustained beneficial effects following treatment.

In particular, the invention provides a combination treatment for treating or preventing a condition and/or disease in a subject comprising administering to the subject a therapeutically effective amount of at least one DPP-IV inhibitor and at least one gastrin compound to produce beneficial effects, preferably sustained beneficial effects.

The invention also relates to a method of treatment comprising administering a therapeutically effective amount of at least one DPP-IV inhibitor in combination with the administration of at least one gastrin compound which upon administration to a subject with symptoms of diabetes produces beneficial effects, preferably sustained beneficial effects, manifested as reduced blood glucose levels, preferably to about normal levels, and/or increased pancreatic insulin.

In an aspect of the invention therapeutically effective amounts of at least one DPP-IV inhibitor and at least one gastrin compound are combined prior to administration to a subject. In an embodiment, therapeutically effective amounts of one or more DPP-IV inhibitor and one or more gastrin compound are mixed at a physiologically acceptable pH.

In other aspects the invention relates to a method for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or B-cell protection, preferably for the treatment and/or prophylaxis of non-insulin dependent diabetes mellitus and/or impaired glucose tolerance, which method comprises administering at least one DPP-IV inhibitor and at least one gastrin compound to a subject in particular a human being or animal. Furthermore, the invention relates to a method for the treatment and/or prophylaxis as defined above, wherein the disease is hypertension or wherein a diuretic agent has a beneficial effect.

In addition, the invention relates to the use of compounds as defined above for the preparation of medicaments for the treatment and/or prophylaxis of diseases which are associated with DPP-IV such as diabetes, particularly non-insulin dependent diabetes mellitus, impaired glucose tolerance, inflammatory bowel disease, Colitis Ulcerosa, Morbus Crohn, obesity, and/or metabolic syndrome or, B-cell protection, preferably for the treatment and/or prophylaxis of non-insulin dependent diabetes mellitus and/or impaired glucose tolerance. Furthermore, the invention relates to the use as defined above, wherein the disease is hypertension or the use for the preparation of diuretic agents.

In context with the methods and uses defined above, the following diseases relate to particular aspects: diabetes, particularly non-insulin dependent diabetes mellitus, impaired glucose tolerance, obesity, and/or metabolic syndrome or B-cell protection, preferably non-insulin dependent diabetes mellitus and/or impaired glucose tolerance.

In an embodiment, the invention provides a method for stimulating beta cell proliferation in a subject comprising administering a therapeutically effective amount of a composition or conjugate of the invention, or administering in combination a DPP-IV inhibitor and a gastrin compound.

In another embodiment, the invention provides a method for increasing the number and/or size of beta cells in a subject comprising administering a therapeutically effective amount of a composition or conjugate of the invention or administering in combination a DPP-IV inhibitor and a gastrin compound.

In a further embodiment, the invention provides a method for preventing or treating Type I or Type II diabetes comprising administering a therapeutically effective amount of a composition or conjugate of the invention, or administering in combination a DPP-IV inhibitor and a gastrin compound.

In a still further embodiment, the invention provides a method for ameliorating progression of disease or obtaining a less severe stage of disease in a person suffering from Type II diabetes comprising administering a therapeutically effective amount of a composition or conjugate of the invention, or administering in combination a DPP-IV inhibitor and a gastrin compound.

The invention relates to a method of delaying the progression of impaired glucose tolerance or non-insulin requiring Type II diabetes to insulin requiring Type II diabetes comprising administering a therapeutically effective amount of a composition or conjugate of the invention, or administering in combination a DPP-IV inhibitor and a gastrin compound.

The invention also relates to a method of increasing the insulin synthesis capability of a subject comprising administering a therapeutically effective amount of a composition or conjugate of the invention, or administering in combination a DPP-IV inhibitor and a gastrin compound.

The invention further relates to inducing islet neogenesis in a subject comprising contacting islet precursor cells with a DPP-IV inhibitor and a gastrin compound, composition, or conjugate of the invention in a sufficient amount to increase proliferation of islet precursor cells in the subject thereby inducing islet neogenesis.

The invention contemplates a method of expanding a functional beta cell mass of pancreatic islet transplants in a diabetic patient, the method comprising administering to the patient a therapeutically effective amount of a DPP-IV inhibitor and a gastrin compound, or a composition or conjugate of the invention.

In an aspect, the invention provides methods for treating diabetes mellitus in a patient in need thereof by administering a composition comprising a gastrin compound and a DPP-IV inhibitor in an amount sufficient to effect differentiation of the patient's pancreatic islet precursor cells to mature insulin-secreting cells and/or to stimulate insulin synthesis in existing islet cells. The composition in some aspects can be administered systemically or expressed in situ by host cells containing one or more nucleic acid construct in an expression vector wherein the nucleic acid construct comprises a coding sequence for a gastrin compound or a coding sequence for a peptide DPP-IV inhibitor or for both compounds, together with transcriptional and translational regulatory regions functional in pancreatic islet precursor cells.

The invention provides methods for treating cells, preferably cells in culture using a DPP-IV inhibitor and gastrin compound of the invention, or compositions, or conjugates of the invention. The invention also provides cell based treatment methods using a DPP-IV inhibitor and a gastrin compound of the invention, or compositions, or conjugates of the invention. [See PCT/CA03/33595 for a description of general culture and cell based treatment methods.]

In an aspect, the invention relates to a method for expanding and differentiating stem cells or progenitor cells into insulin secreting cells comprising contacting the stem cells or progenitor cells with a DPP-IV inhibitor and a gastrin compound or a composition or conjugate of the invention in sufficient amounts to expand and differentiate stem cells or progenitor cells. The amount of expansion and differentiation may be significantly different compared with that achieved in the absence of the compounds, composition or conjugate, in particular the amount may be significantly greater compared with an amount achieved with a DPP-IV inhibitor or a gastrin compound alone. In an embodiment, the stem cells or progenitor cells are contacted with the compounds, composition, or conjugate in culture. In another embodiment, the stem cells or progenitor cells are contacted with the compounds, composition, or conjugate in a subject. The compounds, composition or conjugate may be administered to a subject before, during, or after implantation of stem cells in the subject to expand and differentiate the stem cells in the subject. The stem cells may be obtained from pancreatic islets, umbilical cords, embryos, or stem cell lines. The method may additionally comprise administering an immunosuppressive agent.

The invention also relates to a method for enhancing proliferation of insulin secreting cells in culture comprising contacting the cells with a DPP-IV inhibitor and a gastrin compound, composition or conjugate of the invention in sufficient amounts to enhance proliferation of the cells. The amount of proliferation may be significantly different compared with that achieved in the absence of the compounds, composition or conjugate.

The invention further relates to a method for sustaining islet cells or precursor cells in culture comprising culturing the cells in the presence of a DPP-IV inhibitor and a gastrin compound, composition, or conjugate of the invention in an amount sufficient to sustain the cells in culture. The cells may be sustained in culture for a significantly longer period of time compared with cells cultured in the absence of the compounds, composition or conjugate, or in the presence of a DPP-IV inhibitor or a gastrin compound alone. Culturing cells in the presence of a DPP-IV inhibitor and a gastrin compound or a composition or conjugate of the invention will be particularly useful in preparing and maintaining cells intended for transplantation.

In an aspect, the invention provides a method of treating a condition and/or disease comprising administering a DPP-IV inhibitor and a gastrin compound, a composition or conjugate of the invention with a plurality of cells to a subject in need thereof to thereby produce a beneficial effect, preferably a sustained beneficial effect.

A method for treating a subject with a condition and/or disease described herein comprises contacting ex vivo a plurality of cells with a DPP-IV inhibitor and a gastrin compound, or a composition or conjugate of the invention of the invention, optionally culturing the cells, and administering the cells to the subject in need thereof.

In embodiments of the aforementioned cell based therapeutic methods the cells are pancreatic ductal cells and the amount of compounds/composition/conjugate used in the method is generally effective to increase the amount of insulin secreting cells in the subject. The cells may be autologous (i.e. from the same subject), or may be from another individual of the same species, or from a different species.

The invention also contemplates a method for treating diabetes in a subject comprising transplanting a pancreatic islet preparation into the subject and administering a therapeutically effective amount of a DPP-IV inhibitor and a gastrin compound, or a composition or conjugate of the invention.

In the cell based methods of the invention the number of cells administered to an individual afflicted with a condition and/or disease will vary according to the severity of the condition and/or disease, the mode of administration, and/or the site of administration. Generally a therapeutically effective amount of cells is a safe and effective amount, and in particular an amount necessary to provide one or more beneficial effect, in particular a sustained beneficial effect, or a synergistic effect.

Cells can be administered to subjects using a variety of means apparent to those of skill in the art. Suitable methods include injection of the cells into a target site in a subject. Cells may be inserted into a delivery device to facilitate injection or implantation into the subjects. Examples of delivery devices include tubes, e.g., catheters, for injecting cells and fluids into the body of a subject. Cells can be prepared for delivery in a variety of different forms. For example, the cells may be suspended in a solution or gel, or mixed with a pharmaceutically acceptable carrier, excipient, or diluent in which the cells remain viable. Pharmaceutically acceptable carriers, excipients, and diluents include saline, aqueous buffer solutions, solvents and/or dispersion media. The use of such carriers and diluents is well known in the art. The solution is generally sterile, and will often be isotonic. A solution of cells is preferably selected that is stable under the conditions of manufacture and storage and preserved against the contaminating action of microorganisms through the use of, for example, parabens, chlorobutanol, phenol, scorbic acid, thimerosal, and the like.

Modes of administration of cells include without limitation systemic intracardiac, intracoronary, intravenous, intradermal, or intra-arterial injection and injection directly into the tissue or organ at the intended site of activity, or in proximity to the site of activity. A cell preparation can be administered by any convenient route, for example by infusion or bolus injection and can be administered together with other biologically active agents. Administration in some aspects is preferably systemic. A cell preparation can be administered by any convenient route, for example by infusion or bolus injection and can be administered together with other biologically active agents.

Methods of the invention may further comprise measuring or monitoring one or more of the following markers: blood glucose, serum glucose, blood glycosylated haemoglobin, pancreatic beta cell mass, serum insulin, pancreatic insulin levels, morphometrically determined beta cell mass, amount of insulin secreting cells, and glucose responsiveness of insulin secreting cells.

The invention also contemplates the use of a composition comprising a combination of at least one DPP-IV inhibitor and at least one gastrin compound for the preparation of a medicament providing beneficial effects, preferably sustained beneficial effects in treating a condition and/or disease. In an aspect, the invention relates to the use of a therapeutically effective amount of at least one DPP-IV, and at least one gastrin compound for preparation of a medicament for providing beneficial effects, preferably sustained beneficial effects, in treating a condition and/or disease. In an embodiment the invention provides the use of a DPP-IV inhibitor and a gastrin compound for the preparation of a medicament for increasing (preferably sustained increase) the number and/or size of beta cells in a subject after treatment. In another embodiment the invention provides the use of DPP-IV inhibitor and a gastrin compound for the preparation of a medicament for stimulation (preferably sustained stimulation) of beta cell proliferation after treatment. In a still further embodiment the invention provides the use of a DPP-IV inhibitor and Gastrin for the preparation of a medicament for treatment of a condition and/or disease disclosed herein, especially Type I or Type II diabetes.

The invention additionally provides uses of a pharmaceutical composition and a conjugate of the invention in the preparation of medicaments for beneficial effects, preferably sustained beneficial effects, in the treatment of conditions and/or diseases.

Therapeutic efficacy and toxicity of compounds, compositions and conjugates of the invention may be determined by standard pharmaceutical procedures in cell cultures or with experimental animals such as by calculating a statistical parameter such as the ED₅₀ (the dose that is therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

The compositions of the present invention or fractions thereof typically comprise suitable pharmaceutical diluents, excipients, vehicles, or carriers selected based on the intended form of administration, and consistent with conventional pharmaceutical practices. The carriers, vehicles etc. may be adapted to provide an additive, synergistically effective or therapeutically effective amount of the active compounds. Suitable pharmaceutical diluents, excipients, vehicles, and carriers are described in the standard text, Remington's Pharmaceutical Sciences, Mack Publishing Company. By way of example, for oral administration in the form of a capsule or tablet, the active components can be combined with an oral, non-toxic pharmaceutically acceptable inert carrier such as lactose, starch, sucrose, methyl cellulose, magnesium stearate, glucose, calcium, sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof.

In an aspect of the invention a pharmaceutical composition has a pH from about 7 to 10.

Formulations for parenteral administration of a composition of the invention may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

In an embodiment, a solid form pharmaceutical composition is provided (e.g. tablets, capsules, powdered, or pulverized form) comprising a crystalline or amorphous DPP-IV inhibitor and a crystalline or amorphous gastrin compound.

In another embodiment, the invention relates to a liquid drug formulation comprising pharmaceutically acceptable salts of a DPP-IV inhibitor and a gastrin compound, and to lyophilized drug formulations that can be reconstituted to provide suspensions that are stable and suitable for parenteral administration.

In a particular embodiment, the invention relates to an aqueous composition comprising pharmaceutically acceptable salts of a DPP-IV inhibitor and a gastrin compound, and a solvent system which effects solubilization. The invention also provides a drug comprising an aqueous formulation of pharmaceutically acceptable salts of a DPP-IV inhibitor and a gastrin compound with at least one solubilizer.

A composition of the invention may be sterilized by, for example, filtration through a bacteria retaining filter, addition of sterilizing agents to the composition, irradiation of the composition, or heating the composition. Alternatively, the compounds, conjugates, and compositions of the present invention may be provided as sterile solid preparations e.g. lyophilized powder, which are readily dissolved in sterile solvent immediately prior to use. After pharmaceutical compositions have been prepared, they can be placed in an appropriate container and labelled for treatment of an indicated condition. For administration of a composition of the invention, such labelling would include amount, frequency, and method of administration.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The compositions of the invention and components thereof may comprise soluble polymers as targetable drug carriers.

The compounds, compositions, medicaments, and conjugates of the present invention can be administered by any means that produce contact of the active agent(s) with the agent's sites of action in the body of a subject or patient. The active ingredients can be administered simultaneously or sequentially, and in any order at different points in time, to provide the desired beneficial effects. Each active ingredient may be independently administered any effective number of times, including more than once, as may be indicated by a physician or veterinarian.

The compounds, conjugates and compositions can be formulated for sustained release, for delivery locally or systemically. It lies within the capability of a skilled physician or veterinarian to select a form and route of administration that optimizes the effects of the compositions, conjugates, and treatments of the present invention.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. The compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A particular route of administration is parenteral administration, preferably peripheral parenteral administration. Parenteral administration is generally understood to refer to the injection of a dosage form into the body by a sterile syringe or some other mechanical device such as an infusion pump. For the purpose of the present invention parenteral routes include intravenous, intramuscular, subcutaneous, and intraperitoneal routes of administration. For parenteral administration, the compounds or conjugates described herein may be combined with distilled water at an appropriate pH.

The present invention includes combination treatments providing additive or synergistic activity, delivering an additive or synergistically effective amount, or an amount to provide a therapeutically effective amount of at least one DPP-IV inhibitor and at least one gastrin compound, or a conjugate or composition of the invention. Therefore, pharmaceutical compositions suitable for use in the present invention include compositions wherein the active ingredients are contained in a therapeutically effective amount, more particularly a synergistically effective amount.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a condition can be readily determined by an ordinarily skilled physician or veterinarian. A DPP-IV inhibitor and a gastrin compound or a composition of the invention may be administered in a single daily dose, or the total daily dosage may be administered in divided doses of two, three or four times daily.

A dosage protocol for administration may comprise continuous administration of a DPP-IV inhibitor with intermittent administration of a gastrin compound.

A composition, medicament, or treatment of the invention may comprise a unit dosage of at least one DPP-IV inhibitor and a unit dosage of at least one gastrin compound. A “unit dosage” refers to a unitary i.e. a single dose, which is capable of being administered to a patient, and which may be readily handled and packed, remaining as a physically and chemically stable unit dose comprising either the active agents as such or a mixture with one or more solid or liquid pharmaceutical excipients, carriers, or vehicles.

A composition, medicament, or treatment of the invention may comprise a therapeutically effective suboptimal dosage of a DPP-IV inhibitor and a gastrin compound that are more effective at decreasing or reducing glucose levels for a sustained period following treatment compared with a dosage of either a gastrin compound or DPP-IV inhibitor alone. An improved pharmaceutical composition is also contemplated comprising therapeutically effective suboptimal amounts of a DPP-IV inhibitor and a gastrin compound in a form for chronic or acute therapy of a condition and/or disease, in particular diabetes.

In an aspect, a pharmaceutical composition or treatment is provided comprising at least one DPP-IV inhibitor and at least one gastrin compound in doses that are equal to or at least 1.1, 1.5, 2, 3, 4, 5, 6, 7, 8, 9, or 10 fold lower than the doses of each compound required to provide beneficial effects, preferably sustained beneficial effects, to treat a condition and/or disease.

In an aspect the invention provides a pharmaceutical composition comprising between 0.5 to 6000, 100-1500, 100-6000, 1000-6000, 2000-6000, and 3000-6000 micrograms DPP-IV inhibitor per single unit and 0.5 to 6000, 100-3000, 100-6000, 1000-6000, 2000-6000, and 3000-6000 micrograms gastrin compound per single unit.

In an aspect the invention provides a pharmaceutical composition comprising between 0.01 to 1000, 0.01 to 500, 0.01 to 400, 0.01 to 300, 0.01 to 200, 0.01 to 100, 0.01 to 50, 0.01 to 30, 0.01 to 20, 0.1 to 20, 0.1 to 30, 0.1 to 40, 0.1 to 50, and 0.1 to 60 micrograms/kg/day of a DPP-IV inhibitor and 0.01 to 1000, 0.01 to 500, 0.01 to 400, 0.01 to 300, 0.01 to 200, 0.01 to 100, 0.01 to 50, 0.01 to 30, 0.01 to 20, 0.1 to 20, 0.1 to 30, 0.1 to 40, 0.1 to 50, and 0.1 to 60 micrograms/kg/day of a gastrin compound.

In particular aspects of the invention, the dosage ranges for a gastrin compound are generally about 0.01 micrograms to about 500 micrograms of gastrin compound per kilogram body weight per day, for example, about 0.01 micrograms to about 1 micrograms/kg, about 0.1 micrograms/kg to about 10 micrograms/kg, or about 1 microgram/kg to about 50 micrograms/kg.

In another particular aspect the invention provides a pharmaceutical composition comprising 0.1 to 30, 0.1 to 40, 0.1 to 50, and 0.1 to 60 micrograms/kg/day gastrin compound.

In embodiments of the invention providing sustained beneficial effects, the dosage range for administration of a gastrin compound is 1-30 micrograms/kg body weight, in particular 3-30 micrograms/kg body weight, more particularly 5-20 micrograms/kg body weight.

The ratio of DPP-IV inhibitor to gastrin compound in a composition of the invention may be selected to augment the activity of the DPP-IV inhibitor and/or gastrin compound and to provide beneficial effects, preferably sustained beneficial effects. A DPP-IV inhibitor and a gastrin compound may be in a ratio selected to augment the activity of one or both compounds to produce beneficial effects, in particular a sustained beneficial effect, and/or to produce an additive or synergistic effect. In embodiments, the ratio of a DPP-IV inhibitor to a gastrin compound may be from 1:1 to 1:110, 1:1 to 1:100, 1:1 to 1:75, 1:1 to 1:50, 1:1 to 1:25, 1:1 to 1:10, 1:1 to 1:5, and 1:1. In other particular embodiments, the ratio of a gastrin compound to a DPP-IV inhibitor may be from 1:1 to 1:110, 1:1 to 1:100, 1:1 to 1:75, 1:1 to 1:50, 1:1 to 1:25, 1:1 to 1:10, and 1:1 to 1:5.

A DPP-IV inhibitor may be used in combination with a gastrin compound at therapeutically effective weight ratios of between about 1:1 to 1:150, in particular 1:1 to 1:50. In another embodiment, a gastrin compound may be used in combination with a DPP-IV inhibitor at therapeutically effective weight ratios of between about 1:1 to 1:150, in particular 1:1 to 1:50.

A composition or formulation of the invention may administered to a subject continuously for 2 weeks to 12 months, 2 weeks to 6 months, 2-16 weeks, 2 weeks to 12 weeks, and/or 2-8 weeks, or periodically.

The present invention also includes compositions, conjugates, treatments and methods of the invention in combination with one or more additional therapeutic agents including without limitation immunosuppressive agents, antiobesity agents, antidiabetic agents including without limitation insulin, insulin sensitivity enhancers, glucose lowering agents, insulin secretagogues and insulin signaling pathway modulator, appetite regulating agents, antihypertensive agents, agents for the treatment and/or prevention of complications resulting from or associated with a condition and/or disease, in particular diabetes and obesity, anti-nausea medications, anti-headache medications, and general medications that treat or prevent side effects.

In aspects of the invention, an antidiabetic compound is an insulin signaling pathway modulator, such as inhibitors of protein tyrosine phosphatases (PTPases), non-small molecule mimetic compounds and inhibitors of glutamine-fructose-6-phosphate amidotransferase (GFAT), compounds influencing a dysregulated hepatic glucose production, like inhibitors of glucose-6-phosphatase, inhibitors of fructose-1,6-bisphosphatase, inhibitors of glycogen phosphorylase, glucagon receptor antagonists and inhibitors of phosphoenolpyruvate carboxykinase, pyruvate dehydrogenase kinase inhibitors, insulin sensitivity enhancers, insulin secretion enhancers, alpha-glucosidase inhibitors, inhibitors of gastric emptying, insulin, and alpha 2-adrenergic antagonists. In particular aspects, the additional therapeutic compound is nateglinide, a PPAR compound, repaglinide, metformin, rosiglitazone, pioglitazone, troglitazone, glisoxepid, glyburide, glibenclamide, acetohexamide, chloropropamide, glibornuride, tolbutamide, tolazamide, glipizide, carbutamide, gliquidone, glyhexamide, phenbutamide, tolcyclamide, glimepiride and gliclazide, a peroxisome proliferator-activated receptor α compound, or a pharmaceutically acceptable salt of such a compound.

In aspects of the invention the additional therapeutic agent is a PPAR compound.

Since the present invention relates to a method of treatment comprising a combination of active agents which may be administered separately or as conjugates, the invention also provides a kit comprising at least one DPP-IV inhibitor and at least one gastrin compound, a pharmaceutical composition or conjugate in kit form. The invention also relates to a pharmaceutical kit comprising one bottle with a DPP-IV inhibitor and another bottle with a gastrin compound in one box. A kit may comprise a package which houses a container which contains a conjugate or composition of the invention and also houses instructions for administering the conjugate or composition to a subject.

In embodiments of the invention, a pharmaceutical pack or kit is provided comprising one or more containers filled with one or more of the ingredients of a pharmaceutical composition of the invention to provide a beneficial effect, in particular a sustained beneficial effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

In an aspect, the invention relates to a “kit-of-parts”, for example, the components, a DPP-IV inhibitor in free or pharmaceutically acceptable salt form and gastrin compound in free or pharmaceutically acceptable salt form, can be dosed independently or by use of different fixed combinations with distinguished amounts of the components, i.e., at different time points or simultaneously. Parts of a kit may be administered simultaneously or chronologically staggered, i.e., at different points in time and with equal or different time intervals for any component of a kit. Time intervals can be selected such that the effect on the condition and/or disease in the combined use of the parts is larger than the effect that would be obtained by use of only any one of the components.

The present invention thus also relates to a kit of parts comprising (a) an amount of a DPP-IV inhibitor or a pharmaceutically acceptable salt thereof in a first unit dosage form; (b) an amount of at least one gastrin compound or a pharmaceutically acceptable salt thereof, in the form of two or more separate units of the components (a) and (b).

The invention furthermore relates to a commercial package comprising a combination or composition according to the present invention together with instructions for simultaneous, separate or sequential use.

In an aspect a commercial package comprising as active ingredients at least one DPP-IV inhibitor and at least one gastrin compound is provided in the form of two or more separate units of the components, together with instructions for its simultaneous, separate or sequential use, or any combination thereof, in the delay of progression or treatment of a condition and/or disease disclosed herein.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Example 1

### Effects of Gastrin in Combination with a DPP-IV Inhibitor in Acutely-Diabetic NOD Mice

This example describes methods and compositions for reversing diabetes in diabetic NOD mice by stimulating β-cell neogenesis in vivo following systemic treatment with a DPP-IV inhibitor and gastrin. Female NOD mice ages 12-16 weeks will be treated for 18 days only with vehicle (PBS), a DPP-IV inhibitor, or a DPP-IV inhibitor and gastrin, by injection intraperitoneally twice daily within 2 days after diabetes onset. Onset of diabetes will be determined by fasting blood glucose (FBG) levels (9-15 mM compared with normal FBG<6.0 mM). The mice will be monitored daily for urine glucose and weekly for FBG levels.

At the start of the treatments, fasting blood glucose levels will be measured which may generally be in the range 11-14 mM. After 18 days of treatment therapy will be stopped and FBG will be monitored weekly for additional six weeks.

A short course of a combined DPP-IV inhibitor and gastrin treatment to diabetic NOD mice are expected to normalize hyperglycemia to effectively treat the diabetes. The combination may have a prolonged effect on fasting blood glucose levels indicating a stimulation of beta cell neogenesis and insulin production.

### Example 2

### Effects of Gastrin (G1) in Combination with a DPP-IV Inhibitor in Acutely-Diabetic NOD Mice

### Objective:

NOD mice spontaneously develop insulin-dependent diabetes as a result of autoimmune destruction of pancreatic islet β-cells. This study will be aimed at correcting diabetes in NOD mice by regenerating islet β-cells using a DPP-IV Inhibitor and gastrin (G1).

### Method:

Female NOD mice ages 12-16 weeks will be treated for 18 days only, with vehicle (PBS) or with a DPP-IV inhibitor in combination with Gastrin (G1) by intraperitoneal injection (i.p.) Animals will be injected for 18 days, twice daily, within 2 to 5 days after diabetes onset. The fasting blood glucose (FBG) levels are generally about 9-15 mM at diabetes onset (normal FBG <6.0 mM). The mice will be monitored daily for urine glucose levels and weekly for FBG levels during the treatment, and for an additional 6 weeks after the treatment is stopped. The pancreatic insulin levels will be determined in each group as well as histological analysis of the pancreatic tissue will be performed. Pancreatic tissues will be fixed and stained for insulin producing cells. The beta cell mass will be determined by morphometric analysis.

A combination of a DPP-IV inhibitor and a gastrin compound are anticipated to be effective in restoring normal blood glucose levels even after 6 weeks post-treatment; restore pancreatic insulin content from the low levels measured after diabetes onset and before treatment to a level similar to that measured in normoglycemic mice; and increase beta cell mass in NOD mice to near normal levels.

### Example 3

### Modified gastrin compounds/conjugates of PCT/CA03/01778 in Combination with a DPP-IV Inhibitor in Preventing Diabetes Progression in Nod Mice with Recent Onset Diabetes

The effect of treatment by a combination of a DPP-IV inhibitor and unmodified gastrin and a DPP-IV inhibitor and modified gastrin compounds/conjugates will be examined in NOD mice with recent onset diabetes, to determine whether administration of both a DPP-IV inhibitor and gastrin prevents severe hyperglycemia as well as increases pancreatic insulin content in NOD mice with recent-onset diabetes. Gastrin compounds/conjugates to be used are as follows: Compound A—gastrin as synthetic human gastrin I having 17 amino acid residues with a Leu residue at amino acid position 15, Compound B—gastrin as synthetic human gastrin I having 2-17 amino acid residues, Compound C gastrin as synthetic human gastrin I having 2-17 amino acid residues with a HSA polymer linked via (GA)₅ (i.e. Gly-Ala-Gly-Ala-Gly-Ala-Gly-Ala-Gly-Ala).

Non-obese diabetic (NOD) female mice, ages 12-14 weeks, will be monitored for development of onset of diabetes (fasting blood glucose >8.0 to 15 mmol/l), and within 48 hours after onset of symptoms, four groups of mice will each be treated as follows: one group will be treated with vehicle only; and the other group will be administered a DPP-IV inhibitor, and the remaining groups will be treated with a combination of a DPP-IV inhibitor and gastrin compound, each treatment administered via the intraperitoneal route daily.

Therapy will be administered for 14 days to 18 days. Animals will be monitored weekly for fasting blood glucose (FBG) levels. FBG levels will be measured at about 12 hours after food has been withdrawn, and 24 hours after the last peptide or vehicle injection. Upon cessation of therapy, all mice will be monitored for FBG levels for the next 4 weeks (weeks 2-6) so as to determine whether prevention of hyperglycemia persisted after termination of therapeutic treatment. At 14 days to 18 days treatment will be stopped.

The protocol includes sampling of these mice for data again at 6 weeks, and blood collecting blood for assay of FBG and plasma C-peptide, and sacrificing the mice for pancreatic insulin determinations and scoring of islet inflammation (insulitis). From the outset of treatment, mice will neither receive insulin-replacement treatment nor immunosuppression. The following parameters will be assessed: survival rates, pancreatic insulin levels, presence of islet inflammation and fasting blood glucose levels.

A DPP-IV inhibitor in combination with a modified gastrin compounds/conjugates with longer half lives may provide enhanced reduction of blood glucose levels in diabetic animals.

### Example 4

### Combination Therapy with Gastrin and a DPP-4 Inhibitor in Diabetic NOD Mice

Nonobese diabetic (NOD) mice, an animal model for human type 1 (autoimmune) diabetes were entered into the study within ˜1 week of diabetes onset (blood glucose ≧8 mmol/l) and with a blood glucose of 10-15 mmol/l. These acutely diabetic NOD mice were allocated into 10 groups of 8 mice per group and treated as shown in the table below.

| GROUP | TREATMENT |
|---|---|
| 1 | 0 (baseline) |
| 2 | Vehicle for DDP-4 inhibitor |
| 3 | Vehicle for gastrin |
| 4 | DDP-4 inhibitor 10 mg/kg |
| 5 | DPP-4 inhibitor 50 mg/kg |
| 6 | DPP-4 inhibitor 250 mg/kg |
| 7 | Gastrin 15 ug/kg |
| 8 | Gastrin 15 ug/kg + DPP-4 inhibitor 10 mg/kg |
| 9 | Gastrin 15 ug/kg + DPP-4 inhibitor 50 mg/kg |
| 10 | Gastrin 15 ug/kg + DPP-4 inhibitor 250 mg/kg |

The DPP-4 inhibitor administered was (1-[[3-hydroxy-1-adamantyl)amino]acetyl]-2-cyano-(S)-pyrrolidine (Vildagliptin, Galvus®), and its vehicle is 0.5% carboxymethylcellulose and 0.2% Tween −80. DPP-4 inhibitor and vehicle were given once daily by gavage for 4 weeks. The gastrin administered in the study was human gastrin I [17 leu 15], and its vehicle is 100 mmol/l NaCl and 50 mmol/l NaPO₄, PH 7.4. Gastrin and its vehicle were injected s.c. twice daily for 4 weeks. The mice were monitored weekly for blood glucose levels and body weights. All treatments were stopped after 4 weeks and the mice were monitored weekly for blood glucose levels and body weight for an additional 4 weeks. At study week 8, (i.e. after 4 weeks on treatments+4 weeks off treatments), the mice were sacrificed and blood and pancreata were collected for the following studies:
1. Blood glucose was taken from the tail prior to sacrifice and was measured by glucometer (Ascencia Elite, Bayer).
2. Total glycolated hemoglobin was measured by HPLC (Bio-Rad)
3. Plasma C-peptide was measured by RIA (mouse C— peptide specific, Linco)
4. Pancreatic insulin content was measured by RIA (mouse insulin specific, Linco)

Mice from any group that developed severe hyperglycemia (blood glucose >30 mmol/1) and weight loss before 8 weeks of the study were sacrificed and blood and pancreata were collected and examined as described above.

The results are shown in Table 2. A DPP-IV inhibitor in combination with a gastrin provides increased reduction of blood glucose levels to normal levels in diabetic animals compared to the DPP-IV inhibitor or gastrin alone

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

TABLE 1

| DPP-IV inhibitor | Source/Reference |
|---|---|
| Siagliptin (MK-0431) [(2R)-4-oxo-4-(3- | Merck & Co. |
| [trifluoromethyl]-5,6- | EP 1412357, WO 03/04498, U.S. Pat. |
| dihydro[1,2,4]triazolo[4,3-a]pyrazin-7[8H]- | No. 6,699,871, and, Weber et al, |
| yl)-1-(2,4,5-trifluorophenyl)butan-2-amine]) | Diabetes (2004) 53(Suppl. 2): A151, |
| | 633-P (Abstract) U.S. Pat. No. 2003100563 |
| | Villhauer et al, J Med Chem (2003) |
| | 46: 2774-2789 |
| Vildagliptin (Galvus ®) (LAF237) | Novartis |
| | U.S. Pat. No. 6,166,063, WO |
| | 00/34241, EP 1137635, and JP |
| | 2002531547 |
| 1-[[[2-[(5-cyanopyridin-2- | Novartis |
| yl)amino]ethyl]amino] acetyl]-2-cyano-(S)- | WO 98/19998 and JP 2000511559, |
| pyrrolidine (NVP DPP728) | Villhauer et al, J Med Chem (2002) |
| | 45: 2362-2365 |
| 823093 | GlaxoSmithKline |
| 815541 | |
| 825964 | |
| PSN9301 | OSI Pharma |
| | Probiodrug Prosidion |
| SYR322 | Takeda/PPD |
| Saxagliptin (BM-477118) | Bristol-Myers Squibb |
| SYR619 | Takeda/PPD |
| TA-6666 | Tanabe |
| TS-021 | TaishoPharmaceutical Co., Ltd |
| | Eli Lilly and Company |
| ALS 2-0426, | Alantos |
| PHX1149 | Phenomix |
| SSR 162369 | Sanofi-Aventis |
| DPP-IV Inhibitors | Santhera |
| TRC-8XXX | Torrent |
| 3-(L-Isoleucyl)thiazolidine (isoleucine- | JP 2001510442, WO 97/40832, U.S. |
| thiazolidide) | Pat. No. 6,303,661, and DE 19616486 |
| | Pederson et al, Diabetes (1998) |
| | 47: 1253-1258 |
| DPP-IV inhibitors | WO 01/52825 (Novartis) |
| 4-pyrimidone derivatives | WO 05/016911 |
| Hexahydrodiazepinones | WO 05/011581 |
| Pyrido-2,1-A-isoquionline | WO 05/000848 |
| hexahydropyridoisoquinolines | WO 05/000846 |
| 1H-imidazo′4,5-dipyridazines | WO 04/108730 |
| DPP-IV inhibitors | WO 05/056013 |
| DPP-IV inhibitors | WO 01/62266 |
| DPP-IV inhibitors | WO 05/075426 |
| DPP-IV inhibitors | WO 05/058849 |
| DPP-IV inhibitors | WO 05/056541 |
| DPP-IV inhibitors | WO 05/049022 |
| Azolidine carbonitriles | WO 05/033106 |
| DPP-IV inhibitors | WO 05/033099 |
| DPP-IV inhibitors | WO 05/030751 |
| 2-cyanopyrrolidine derivatives | WO 04/099185 |
| Substituted azetidines | WO 04/071454 |
| 2-cyanopyrrolidine derivatives | WO 04/048352 |
| DPP-IV inhibitors | WO 04/048352 |
| Hemisuccinate salts | WO 04/033455 |
| Succinate salts of heterocyclic DPP-IV | WO 04/033455 |
| inhibitors | |
| Phenacyl xanthine derivatives of DPP-IV | WO 04/018467 |
| inhibitors | |
| Pyrido[2,1-A] isoquinoline derivatives | WO 03/055881 and EP 1461337 |
| DPP-IV inhibitors | WO 97/40832 |
| DPP-IV inhibitors | WO 98/19998 |
| Heterocyclic amide compounds | WO 03/000180 (Merck & Co.) |
| DPP-IV inhibitors | WO 03/000181 (Merck & Co.) |
| N-(N′-substituted glycyl)-2-cyano | WO 98/19998 |
| pyrrolidines (e.g., Example 1 DPP728 and | |
| LAF237) | |
| N-substituted adamantyl-amino-acetyl-2- | WO 00/34241 |
| cyano-pyrrolidines | |
| Amino acid 2-cyanopyrrolidine amides (e.g., | WO 95/15309 |
| FE-999011) | |
| DPP-IV inhibitors | WO 01/72290 |
| DPP-IV inhibitors | WO 01/52825 |
| DPP-IV inhibitors | WO 03/002553 |
| praline boronic esters | WO 93/10127 |
| DPP-IV inhibitors | WO 99/61431 |
| Sulphostin | WO 99/25719 |
| N-substituted 4- to 8-membered heterocyclic | WO 99/38501 |
| rings | |
| phosphoric compounds | WO 99/46272 |
| DPP-IV prodrugs and inhibitors of the form | WO 99/67278 |
| A-B-C where C is either a stable or unstable | |
| inhibitor of DPP-IV | |
| DPP-IV prodrugs and inhibitors of the form | WO 99/67279 |
| A-B-C where C is either a stable or unstable | |
| inhibitor of DPP-IV | |
| N-Peptidyl-O-aroyl hydroxylamines | WO 2004/052362 and |
| | PCT/EP2003/013963 |
| Heterocyclic derivatives | WO 05/026148 and U.S. Pat. No. 20050065145 |
| Hexahydrodiazepinone derivatives | WO 04/037169 and U.S. Pat. No. 7,101,871 |
| | (Merck & Co. Inc.) |
| Glycine nitrile compounds | WO 04/037181 and U.S. Pat. No. 6995180 |
| Imidazo-pyridinones and imidazo- | WO 04/050658 and U.S. Pat. No. 7109192 |
| pyridazinones | (Boehringer Ingelheim) |
| Dipeptidyl peptidase IV inhibitors | WO 05/037779A3 (IMTM) |
| Beta-amino heterocyclic compounds | WO 04/032836 and U.S. Pat. No. 20060014953 |
| Crystalline anhydrate polymorphs of | WO 05/020920 |
| dihydrogen phosphate salt of (2R)-4-oxo-4-(3- | |
| (trifluoromethyl)-5,6- | |
| dihydro(1,2,4)triazolo(4,3-a)pyrazin-7(8H-yl)- | |
| 1-(2,4,5-trifluorophenyl)butan-2-amine | |
| Adamantylglycine-based inhibitors | WO 05/012249 and U.S. Pat. No. 6995183 |
| New hexahydrodiazepinone derivatives | WO 05/011581 (Merck & Co) |
| 2-(homo)piperazino-3,5-dihydro-imidazol- | WO 05/058901 |
| (4,5-d)-pyridazin-4-one derivatives | |
| 7-butynyl-8-aminoxanthine derivatives | WO 06/029769 |
| 8-(3-amino-piperidino)-7-(but-2-ynyl)- | WO 06/027204 |
| xanthine derivatives | |
| Imidazopyridazinedione derivatives | WO 05/087774 |
| Ketone compounds and cyclic compounds | WO 05/082348 |
| 8-(3-Amino-piperidin-1-yl)xanthine | WO 05/082906 |
| derivatives | |
| Fluorinated cyclic amides, e.g. (2S)-2-amino- | WO 04/046106 (Pfizer) |
| 2-cyclohexyl-1-((3RS)-3-fluoro-pyrrolidin-1- | |
| yl)-ethanone | |
| dipeptidyl peptidase IV inhibitors | WO 03/057666 |
| 2-(homo)piperazino-3,5-dihydro-imidazol- | WO 05/058901 |
| (4,5-d)-pyridazin-4-one derivatives | |
| Hexahydrodiazepinone derivatives | WO 04/037169 (Merck & Co.) |
| Beta-amino heterocyclic compounds | WO 04/032836 (Merck & Co.) |
| Bicyclic imidazole derivatives | WO 05/063750 |
| 1,7,8-trisubstituted xanthine derivatives | WO 04/046148 (Boehringer |
| | Ingelheim) |
| DPP-IV inhibitors in International Patent | WO 2006/090244, WO 2006/078676, |
| Applications | WO 2006/076231, WO 2006/058628, |
| | WO 2006/058064, WO 2006/040625, |
| | WO 2006/100181, WO 2006/097175, |
| | WO 2006/039325, WO 2006/023750, |
| | WO 2005/113510, WO 2006/013104, |
| | WO 2006/011035, WO 2006/000986, |
| | WO 2005/087235, WO 2005/082348, |
| | WO 2005/082849, WO 2005/079795, |
| | WO 2005/075426, WO 2005/072530, |
| | WO 2005/063750, WO 2005/058849, |
| | WO 2005/049022, WO 2005/047297, |
| | WO 2005/044195, WO 2005/042488, |
| | WO 2005/040095, WO 2005/037828, |
| | WO 2005/037779, WO 2005/034940, |
| | WO 2005/033099, WO 2005/032590, |
| | WO 2005/030751, WO 2005/030127, |
| | WO 2005/026148, WO 2005/025554, |
| | WO 2005/023762, WO 2005/020920, |
| | WO 2005/056003, WO 2005/019168, |
| | WO 05/12312, WO 05/12308, |
| | WO 05/12249, WO 05/11581, |
| | WO 05/09956, WO 05/03135, |
| | WO 05/00848, WO 05/00846, |
| | WO 04/112701, |
| | WO 04/111051, WO 04/111041, |
| | WO 04/110436, WO 04/110375, |
| | WO 04/108730, WO 04/104216, |
| | WO 04/104215, WO 04/103993, |
| | WO 04/103276, WO 04/99134, |
| | WO 04/96806, WO 04/92128, |
| | WO 04/87650, WO 04/87053, |
| | WO 04/85661, WO 04/85378, |
| | WO 04/76434, WO 04/76433, |
| | WO 04/71454, WO 04/69162, |
| | WO 04/67509, WO 04/64778 |
| | WO 04/58266, WO 04/52362, |
| | WO 04/52850, WO 04/50022, |
| | WO 04/50658, WO 04/48379, |
| | WO 04/46106, WO 04/43940, |
| | WO 04/41820, WO 04/41795, |
| | WO 04/37169, WO 04/37181, |
| | WO 04/33455, WO 04/32836, |
| | WO 04/20407, WO 04/18469, |
| | WO 04/18468, WO 04/18467, |
| | WO 04/14860, WO 04/09544, |
| | WO 04/07468, WO 04/07446, |
| | WO 04/04661, WO 04/00327, |
| | WO 03/106456, WO 03/104229, |
| | WO 03/101958, WO 03/101448, |
| | WO 03/99279, WO 03/95425, |
| | WO 03/84940, WO 03/82817, |
| | WO 03/80633, WO 03/74500, |
| | WO 03/72556, WO 03/72528, |
| | WO 03/68757, WO 03/68748, |
| | WO 03/57666, WO 03/57144, |
| | WO 03/55881, WO 03/45228, |
| | WO 03/40174, WO 03/38123, |
| | WO 03/37327, WO 03/35067, |
| | WO 03/35057, WO 03/24965, |
| | WO 03/24942, WO 03/22871, |
| | WO 03/15775, WO 03/04498, |
| | WO 03/04496, WO 03/02530, |
| | WO 03/02596, WO 03/02595, |
| | WO 03/02593, WO 03/02553, |
| | WO 03/02531, WO 03/00181, |
| | WO 03/00180, WO 03/00250, |
| | WO 02/83109, WO 02/83128, |
| | WO 02/76450, WO 02/68420, |
| | WO 02/62764, WO 02/55088, |
| | WO 02/51836, WO 02/38541, |
| | WO 02/34900, WO 02/30891, |
| | WO 02/30890, WO 02/14271, |
| | WO 02/02560, WO 01/97808, |
| | WO 01/96295, WO 01/81337, |
| | WO 01/81304, WO 01/68603, |
| | WO 01/55105, WO 01/52825, |
| | WO 01/34594, WO 00/71135, |
| | WO 00/69868, WO 00/56297, |
| | WO 00/56296, WO 00/34241, |
| | WO 00/23421, WO 00/10549, |
| | WO 99/67278, WO 99/62914, |
| | WO 99/61431, WO 99/56753, |
| | WO 99/25719, WO 99/16864, |
| | WO 98/50066, WO 98/50046, |
| | WO 98/19998, WO 98/18763, |
| | WO 97/40832, WO 95/29691, |
| | WO 95/15309, WO 93/10127, |
| | WO 93/08259, WO 91/16339 |
| DPP-IV inhibitors in US Patent Applications | 200601452585, 20060135512, |
| | 2006021168, |
| | 2006021062, 2005007533, |
| | 2005007070, |
| | 2005059724, 2005059716, |
| | 2005043292, |
| | 2005038020, 2005032804, |
| | 2005004205, |
| | 2005120494, 2005121089, |
| | 20050065145, |
| | 20050065148, 2005121131, |
| | 20050192324, |
| | 20050070535, 20050065144, |
| | 2005007053, |
| | 2005123685, 2004259903, |
| | 2004259902, |
| | 2004259883, 2004254226, |
| | 2004242898, |
| | 2004229926, 2004180925, |
| | 2004176406, |
| | 2004138214, 2004116328, |
| | 2004110817, |
| | 2004106656, 2004097510, |
| | 2004087587, |
| | 2004082570, 2004077645, |
| | 2004072892, |
| | 2004063935, 2004034014, |
| | 2003232788, |
| | 2003225102, 2003216450, |
| | 2003216382, |
| | 2003199528, 2003195188, |
| | 2003162820, |
| | 2003149071, 2003134802, |
| | 2003130281, |
| | 2003130199, 2003125304, |
| | 2003119750, |
| | 2003119738, 2003105077, |
| | 2003100563, |
| | 2003087950, 2003078247, |
| | 2002198205, |
| | 2002183367, 2002103384, |
| | 2002049164, |
| | 2002006899 |
| DPP-IV inhibitors in US Patents | 7,101,871, 6,869,947, 6,867,205, |
| | 6,861,440, |
| | 6,849,622, 6,803,357, 6,800,650, |
| | 6,727,261, |
| | 6,716,843, 6,710,040, 6,645,995, |
| | 6,617,340, |
| | 6,699,871, 6,573,287, 6,432,969, |
| | 6,395,767, |
| | 6,380,398, 6,242,422, 6,166,063, |
| | 6,100,234, |
| | 6,040,145 |
| 3,3,4,4-tetrafluoropyrrolidine | U.S. Pat. No. 6,8123,50 |
| W (substituted glycyl)-4-cyano pyrrolidines | U.S. Pat. No. 6,110,949 |
| Glutaminyl based DPIV inhibitors | U.S. Pat. No. 6,946,480 |
| Valyl-thiazolidide and valyl-pyrrolidide | U.S. Pat. No. 6,548,481 |
| Xanthine derivatives | U.S. Pat. No. 7,074,798 (Eisai Co., Ltd) |
| Alpha amino acid derivatives | U.S. Pat. No. 6,706,742 |
| Glutaminyl based DPIV inhibitors | U.S. Pat. No. 6,946,480 |
| N-valyl prolyl, O-benzoyl hydroxylamine | U.S. Pat. No. 6,303,661 |
| Valyl-thiazolidide and valyl-pyrrolidide | U.S. Pat. No. 6548481 |
| val-pyr, val-thiazolidide, isoleucyl- | DE 19616 486 A1 |
| thiazolidide, isoleucyl-pyrrolidide, and fumar | |
| salts of isoleucyl-thiazolidide and isoleucyl- | |
| pyrrolidide | |
| DPP-IV inhibitors in Canadian Patent | CA 2466870, CA 2433090, CA |
| Applications | 2339537, |
| | CA 2289125, CA 2289124, CA |
| | 2123128 |
| DPP-IV inhibitors in European Patent | EP 1613304, EP 1604989, EP |
| Applications | 1604980, |
| | EP 1604662, EP 1598341, EP |
| | 1593671, |
| | EP 1521148, EP 1702916, EP |
| | 1682540, |
| | EP 1664031, EP 1338595, EP |
| | 1638970, |
| | EP 1638968, EP 1541148, EP |
| | 1517907, |
| | EP 1513808, EP 1492777, EP |
| | 1490335, |
| | EP 1489088, EP 1480961, EP |
| | 1476435, |
| | EP 1476429, EP 1469873, EP |
| | 1465891, |
| | EP 1463727, EP 1461337, EP |
| | 1450794, |
| | EP 1446116, EP 1442049, EP |
| | 1441719, |
| | EP 1426366, EP 1412357, EP |
| | 1406873, |
| | EP 1406872, EP 1406622, EP |
| | 1404675, |
| | EP 1399420, EP 1399471, EP |
| | 1399470, |
| | EP 1399469, EP 1399433, EP |
| | 1399154, |
| | EP 1385508, EP 1377288, EP |
| | 1355886, |
| | EP 1354882, EP 1338592, EP |
| | 1333025, |
| | EP 1304327, EP 1301187, EP |
| | 1296974, |
| | EP 1280797, EP 1282600, EP |
| | 1261586, |
| | EP 1258476, EP 1254113, EP |
| | 1248604, |
| | EP 1245568, EP 1215207, EP |
| | 1228061, |
| | EP 1137635, EP 1123272, EP |
| | 1104293, |
| | EP 1082314, EP 1050540, EP |
| | 1043328, |
| | EP 0995440, EP 0980249, EP |
| | 0975359, |
| | EP 0731789, EP 0641347, EP |
| | 0610317, |
| | EP 0528858 |
| | DE 19834591, DE 19828113, |
| | DE 19823831, DE 19616486, |
| | DE 10333935, DE 10327439, |
| | DE 10256264, DE 10251927, |
| | DE 10238477, DE 10238470, |
| | DE 10238243, DE 10143840, |
| | DD 296075, FR 2824825, FR 2822826 |
| DPP-IV inhibitors in Japanese Patent | JP 2005507261, JP 2005505531, |
| Applications | JP 2005502624, JP 2005500321, |
| | JP 2005500308, JP 2005023038, |
| | JP 2004536115, JP 2004535445, |
| | JP 2004535433, JP 2004534836, |
| | JP 2004534815, JP 2004532220, |
| | JP 2004530729, JP 2004525929, |
| | JP 2004525179, JP 2004522786, |
| | JP 2004521149, JP 2004503531, |
| | JP 2004315496, JP 2004244412, |
| | JP 2004043429, JP 2004035574, |
| | JP 2004026820, JP 2004026678, |
| | JP 2004002368, JP 2004002367, |
| | JP 2003535898, JP 2003535034, |
| | JP 2003531204, JP 2003531191, |
| | JP 2003531118, JP 2003524591, |
| | JP 2003520849, JP 2003327532, |
| | JP 2003300977, JP 2003238566, |
| | JP 2002531547, JP 2002527504, |
| | JP 2002517401, JP 2002516318, |
| | JP 2002363157, JP 2002356472, |
| | JP 2002356471, JP 2002265439, |
| | JP 2001510442, JP 2000511559, |
| | JP 2000327689, JP 2000191616, |
| | JP 1998182613, JP 1998081666, |
| | JP 1997509921, JP 1995501078, |
| | JP 1993508624 |
| DPP-IV inhibitors | George R. Lankas et al, Diabetes |
| | 54: 2988-2994, 2005 |
| N-Peptidyl-O-aroyl hydroxylamines | Mona Patel and Colt (Expert Opinion |
| | Investig Drugs. 2003 Apr; 12(4): 623-33 |
| DPP-IV inhibitors | Diabetes, Vol. 47, pp.1253-1258 (1998 |
| LAF237 | Villhauer et al., J Med Chem (2003) |
| | 46: 2774-2789 |
| DPP-IV Inhibitors | Ahren et al, J Clin Endocrinol Metab |
| | (2004) 89: 2078-2084 |
| NVP-DPP728 | Villhauer et al., J Med Chem (2002) |
| | 45: 2362-2365 |
| NVP-DPP728 | Ahren et al, Diabetes Care (2002) |
| | 25: 869-875 |
| DPP-IV Inhibitors | Caldwell et al., Bioorg Med Chem Lett |
| | (2004) 14: 1265-1268 |
| DPP-IV Inhibitors | Peters et al., Bioorg Med Chem Lett |
| | (2004) 14: 1491-1493 |
| DPP-IV Inhibitors | Edmondson et al., Bioorg Med Chem |
| | Lett (2004) 14: 5151-5155 |
| Valine-pyrrolidide | Deacon et al, Diabetes (1998) |
| | 47: 764769 |
| MK-0431 | Weber et al, Diabetes (2004) 53 |
| | (Suppl. 2): A151, 633-P (Abstract) |
| DPP-IV Inhibitors | Abe et al., J Nat Prod (2004) 67: 999-1004 |

TABLE 2
Effects of a DPP - 4 Inhibitor ± Gastrin on Blood Glucose in Nonobese
Diabetic (NOD) Mice - An Animal Model for Human Type 1 Diabetes

| | | | Blood Glucose (mmol/l) | | | | |
|---|---|---|---|---|---|---|---|
| | | # of | 0 | | | | |
| Group | Treatment | mice | weeks | 1 weeks | 2 weeks | 3 weeks | 4 weeks |
| 1 | vehicle | 7 | 9.0 ± 0.4 | 9.8 ± 0.6 | 23.7 ± 3.4 | 28.7 ± 3.6 | 23.6 ± 4.8^{a} |
| | (methylcellulose) | | | | | | |
| 2 | DPP - 4 inhibitor | 8 | 9.7 ± 0.4 | 9.2 ± 0.3 | 8.7 ± 0.5 | 7.4 ± 0.4 | 7.8 ± 1.1 |
| | 250 mg/kg | | | | | | |
| 3 | vehicle | 6 | 9.3 ± 0.5 | 11.2 ± 1.0 | 20.1 ± 4.0 | 25.1 ± 3.6 | 32.1 ± 1.7^{b} |
| | (PBS) | | | | | | |
| 4 | Gastrin | 9 | 9.5 ± 0.4 | 8.2 ± 0.4 | 7.2 ± 0.4 | 9.0 ± 2.3 | 11.9 ± 3.8 |
| | 15 ug/kg | | | | | | |
| 5 | DPP - 4 inhibitor + | 8 | 9.6 ± 0.3 | 9.2 ± 0.2 | 5.7 ± 0.3 | 5.6 ± 0.3 | 5.9 ± 0.3 |
| | Gastrin | | | | | | |

NOD female mice, age 10-12 weeks, were treated within 1 week of diabetes onset (blood glucose >7.5 mmol/l) with: vehicle (0.5% methylcellulose + 0.2% Tween 80 in water) (Group 1) or a DPP - 4 inhibitor in this methylcellulose vehicle (Group 2), both treatments given by oral gavage once daily; a PBS vehicle (Group 3) or gastrin in this PBS vehicle (Group 4), both treatments given by subcutaneous injection twice daily; and the DPP - 4 inhibitor + gastrin (Group 5).
^{a}only 4 of the original 7 mice survived to 4 weeks
^{b}only 4 of the original 6 mice survived to 4 weeks

1. A pharmaceutical composition comprising therapeutically effective amounts of a DPP-IV inhibitor and a gastrin compound, and a pharmaceutically acceptable carrier, excipient, or vehicle.

2-10. (canceled)

11. A pharmaceutical composition according to claim 1 wherein the DPP-IV inhibitor and the gastrin compound are present in doses that are at least about 1.1 to 1.4, 1.5, 2, 3, 4, 5, 6, 7, 8, 9, or 10 fold lower than the doses of each compound alone required to treat diabetes.

12. (canceled)

13. A pharmaceutical composition according to claim 1 comprising a synergistically effective amount of the DPP-IV inhibitor and the gastrin compound in a pharmaceutically acceptable excipient, carrier, or vehicle.

14-20. (canceled)

21. A pharmaceutical composition according to claim 1 wherein the gastrin compound is a compound of the formula Z-Ym-Xn-AA1-AA2-AA3-AA4-AA5-AA6, wherein AA1 is Tyr or Phe, AA2 is Gly, Ala, or Ser, AA3 is Trp, Val, or Ile, AA4 is Met or Leu, AA5 is Asp or Glu, and AA6 is Phe or Tyr which is optionally amidated; Z is an optional carrier, preferably a polymer, more preferably a protein; Ym is an optional spacer region comprising m amino acid residues of a small neutral amino acid including but not limited to serine and alanine, and X is any consecutive portion of residues 1-28 of SEQ ID NO: 1 or 2, or residues 1-11 of SEQ ID NO. 3 or 4, preferably AA1-AA2-AA3-AA4-AA5-AA6 is Tyr-Gly-Trp-Met-Asp-Phe or Tyr-Gly-Trp-Leu-Asp-Phe.

22. A pharmaceutical composition according to claim 21 wherein the gastrin compound is gastrin 17 or analogs and derivatives thereof.

23. A pharmaceutical composition according to claim 21 wherein the gastrin compound is synthetic human gastrin I having 17 amino acid residues with a Leu residue at amino acid position 15 [SEQ ID NO. 4].

24-25. (canceled)

26. A pharmaceutical composition according to claim 21 wherein the DPP-IV inhibitor is selected from the group consisting of sitagliptin, vildagliptin, PSN9301, saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

27. A pharmaceutical composition according to claim 21 wherein the DPP-IV inhibitor is sitagliptin, vildagliptin, PSN9301 or saxagliptin.

28. A method for treating or preventing diabetes in a subject comprising administering to the subject therapeutically effective amounts of a DPP-IV inhibitor and a gastrin compound to produce a beneficial effect.

29-30. (canceled)

31. A method according to claim 28 wherein therapeutically effective amounts of the DPP-IV inhibitor and the gastrin compound are combined prior to administration to the subject.

32. A method according to claim 28 wherein therapeutically effective amounts of the DPP-IV inhibitor and the gastrin compound are administered to the subject sequentially.

33. A method according to claim 28 wherein the therapeutically effective amounts of a DPP-IV inhibitor and a gastrin compound are synergistically effective amounts.

34-38. (canceled)

39. A method for inducing islet neogenesis in a subject comprising contacting islet precursor cells with a DPP-IV inhibitor and a gastrin compound, or a composition of any preceding claim in a sufficient amount to increase proliferation of islet precursor cells in the subject thereby inducing islet neogenesis.

40-42. (canceled)

43. A method according to claim 28 wherein the gastrin 34 (big gastrin) (SEQ ID NO. 1 or 2), gastrin 17 (little gastrin) (SEQ ID NO. 3 or 14), gastrin 14 (SEQ ID NO. 7), gastrin 8, gastrin 6 (SEQ ID NO. 8 or 9), pentagastrin, or tetragastrin

44. A method according to claim 28 wherein the gastrin compound is a compound of the formula Z-Ym-Xn-AA1-AA2-AA3-AA4-AA5-AA6, wherein AA1 is Tyr or Phe, AA2 is Gly, Ala, or Ser, AA3 is Trp, Val, or Ile, AA4 is Met or Leu, AA5 is Asp or Glu, and AA6 is Phe or Tyr which is optionally amidated; Z is an optional carrier, preferably a polymer, more preferably a protein; Ym is an optional spacer region comprising m amino acid residues of a small neutral amino acid including but not limited to serine and alanine, and X is any consecutive portion of residues 1-28 of SEQ ID NO: 1 or 2, or residues 1-11 of SEQ ID NO. 3 or 4, preferably AA1-AA2-AA3-AA4-AA5-AA6 is Tyr-Gly-Trp-Met-Asp-Phe or Tyr-Gly-Trp-Leu-Asp-Phe.

45. A method according to claim 28 wherein the gastrin compound is gastrin 17 or analogs and derivatives thereof.

46. A method according to claim 28 wherein the gastrin compound is synthetic human gastrin I having 17 amino acid residues with a Leu residue at amino acid position 15 [SEQ ID NO. 4].

47-48. (canceled)

49. A method according to claim 28 wherein the DPP-IV inhibitor is selected from the group consisting of sitagliptin, vildagliptin, PSN9301, saxagliptin, N-(N′-substituted glycyl)-2-cyanopyrrolidines, L-threo-isoleucyl thiazolidine (P32/98), L-allo-isoleucyl thiazolidine, L-threo-isoleucyl pyrrolidine, or L-allo-isoleucyl pyrrolidine.

50. A method according to claim 28 wherein the DPP-IV inhibitor is sitagliptin, vildagliptin, PSN9301 or saxagliptin.

51-61. (canceled)

62. A kit form of a composition as claimed in claim 1.
